(19) United States
(12) Patent Application Publication
(10) Pub. No.: US 2025/0171493 A1
(43) Pub. Date: May 29, 2025

(54) PROCESSES FOR SYNTHESIS OF WITHANOLIDES AND WITHAFERINS AND ANALOGS THEREOF

(71) Applicant: H. LEE MOFFITT CANCER CENTER AND RESEARCH INSTITUTE, INC., Tampa, FL (US)

(72) Inventor: Justin Matthew LOPCHUK, Tampa, FL (US)

(21) Appl. No.: 18/844,216

(22) PCT Filed: Mar. 6, 2023
(86) PCT No.: PCT/US2023/014595

§ 371 Date: Sep. 5, 2024

Related U.S. Application Data
(60) Provisional application No. 63/316,972, filed on Mar. 5, 2022.

(51) Int. Cl. C07J 71/00; C07J 9/00; C07J 33/00

(52) U.S. Cl. CPC C07J 71/001; C07J 9/00; C07J 33/002

(57) ABSTRACT

The present disclosure provides processes for the synthesis of withanolides such as Withanolide A and Withanolide D.

### CROSS-REFERENCE TO RELATED APPLICATIONS

This application claims the benefit of priority to U.S. Provisional Application No. 63/316,972, filed Mar. 5, 2022, the disclosure of which is incorporated herein by reference in its entirety.

### BACKGROUND

Withanolides are a family of over 600 natural products (Misico, et al., Prog. Chem. Org. Nat'l. Prod., 2011, 94:127; Glotter, et al., Nat. Prod. Rep. 1991, 8, 415). Withanolides have a steroidal backbone bearing a δ-lactone and a highly oxidized A,B-ring system. One member of the withanolide family, Withaferin A, was isolated from Withania somnifera and inhibits STAT3 phosphorylation in MDA-MB-231 cells (Lee, et al., Carcinogenesis 2010, 31, 1991). In addition, withaferin A has apparent pleiotropic activity, including antitumor, anti-inflammatory, antistress, antifeedant, and antioxidant activities (Singh, et al., Ann. Biol. Res., 2010, 1, 56; Budhiraja, et al., J. Sci. Ind. Res. 2000, 59, 904). Identified cellular targets of withaferin A include, annexin II, vimentin, HSP90, IKKβ, β-tubulin, NF-κB essential modulator, and AAA+ chaperone p97. Because cysteine residues of these target proteins form covalent bonds at the C3 of withaferin A, the 2-en-1-one moiety seems to be the pharmacophore.

Another withanolide, withacnistin, has been shown to inhibit STAT3 phosphorylation (Sun, et al., Oncogene 2005, 24, 3236; Zhang, et al., Br. J. Cancer 2014, 111, 894). An ethanol Michael adduct of withacnistin at C3 does not inhibit JAK/STAT pathway signaling, suggesting that the A-ring enone, common to withaferin A and withacnistin, is important for inhibition of STAT3 phosphorylation (Sun, et al., Oncogene 2008, 27, 1344). The mechanism by which withanolides inhibit JAK/STAT pathway activity has not been determined.

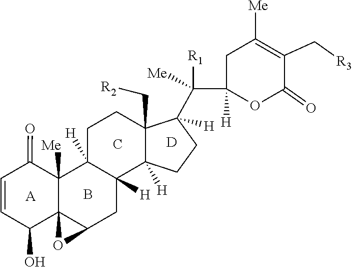

Withaferin A: R¹=H, R²=H, R³=OH
Withacnistin: R¹=H, R²=OAc, R³=H
Withanolide D: R¹=OH, R²=H, R³=H.

Given their activity against various cellular targets, withaferin A and withacnistin are of interest for various therapeutic strategies. Further, it is desirable to create new withanolides to elucidate mechanisms of action of this class of molecules. However, only one total synthesis of withaferin A has been reported, in 33 steps from 3β-hydroxy-22,23-bisnorchol-5-enoic acid, (U.S. Pat. No. 4,193,921; Hirayama, et al., J. Am. Chem. Soc. 1982, 104, 3735; Hirayama, et al., Tetrahedron Lett. 1982, 23, 4725) and no syntheses of withacnistin have been reported. What is thus needed is a flexible, scalable synthetic route to withaferin A, withacnistin, and other withanolides. The compositions and methods disclosed herein address these and other needs.

### SUMMARY

In accordance with the purposes of the disclosed subject matter as embodied and broadly described herein, the disclosed subject matter relates to compositions and methods of making and using said compositions. In a further aspect, the disclosed subject matter relates to withanolides and to methods of making and using withanolides, such as withaferin A, withanolide D, withanolide A, withancnistin, and other analogs.

Additional aspects and advantages of the disclosure will be set forth, in part, in the detailed description and any claims which follow, and in part will be derived from the detailed description or can be learned by practice of the various aspects of the disclosure. The advantages described below will be realized and attained by means of the elements and combinations particularly pointed out in the appended claims. It is to be understood that both the foregoing general description and the following detailed description are exemplary and explanatory only and are not restrictive of the disclosure.

### DESCRIPTION OF DRAWINGS

The accompanying figures, which are incorporated in and constitute a part of this specification, illustrate several aspects described below.

FIG. 1A is a schematic of an exemplary synthesis of withaferin A.

FIG. 1B is a schematic of an exemplary synthesis of a precursor to withaferin A.

FIG. 2 is a schematic of various withanolides that can be prepared according to the disclosed methods.

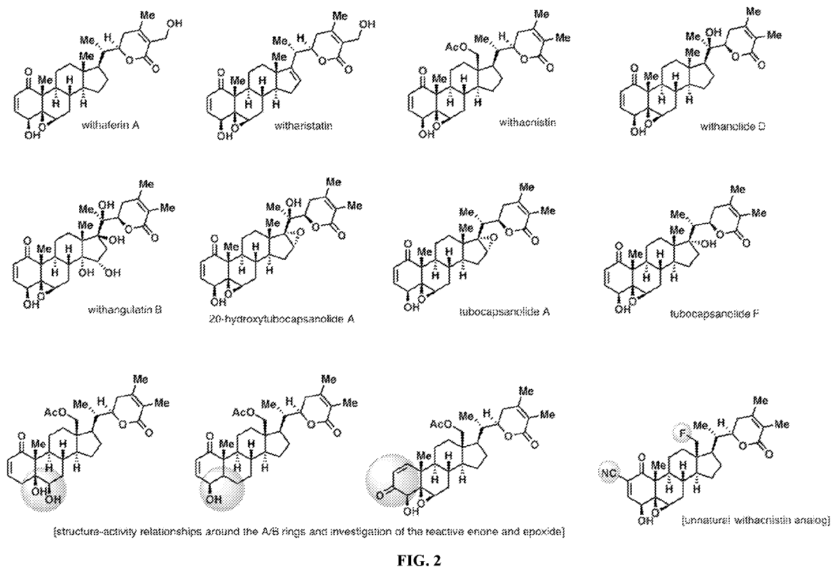

FIGS. 3A and 3B shows biological data for certain withaferin analogs.

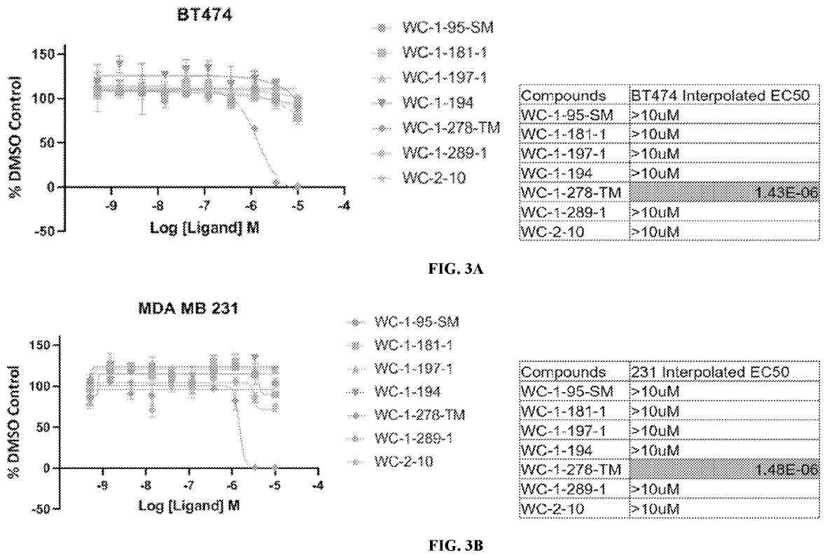

FIG. 4 shows the structures of various withaferin analogs whose biological data are in FIGS. 3A and 3B.

### DETAILED DESCRIPTION

The compounds, compositions, and methods described herein may be understood more readily by reference to the following detailed description of specific aspects of the disclosed subject matter and the Examples and Figures included therein.

Before the present compounds, compositions, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings.

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the component” includes mixtures of two or more such components, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

By “reduce” or other forms of the word, such as “reducing” or “reduction,” is meant lowering of an event or characteristic (e.g., tumor growth, metastasis). It is understood that this is typically in relation to some standard or expected value, in other words it is relative, but that it is not always necessary for the standard or relative value to be referred to. For example, “reduces tumor growth” means decreasing the amount of tumor cells relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

As used herein, “treatment” refers to obtaining beneficial or desired clinical results. Beneficial or desired clinical results include, but are not limited to, any one or more of: alleviation of one or more symptoms (such as tumor growth or metastasis), diminishment of extent of cancer, stabilized (i.e., not worsening) state of cancer, preventing or delaying spread (e.g., metastasis) of the cancer, delaying occurrence or recurrence of cancer, delay or slowing of cancer progression, amelioration of the cancer state, and remission (whether partial or total).

The term “patient” preferably refers to a human in need of treatment with an anti-cancer agent or treatment for any purpose, and more preferably a human in need of such a treatment to treat cancer, or a precancerous condition or lesion. However, the term “patient” can also refer to non-human animals, preferably mammals such as dogs, cats, horses, cows, pigs, sheep and non-human primates, among others, that are in need of treatment with an anti-cancer agent or treatment.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

### Chemical Definitions

As used herein, the term “composition” is intended to encompass a product comprising the specified ingredients in the specified amounts, as well as any product which results, directly or indirectly, from combination of the specified ingredients in the specified amounts.

References in the specification and concluding claims to parts by weight of a particular element or component in a composition denotes the weight relationship between the element or component and any other elements or components in the composition or article for which a part by weight is expressed. Thus, in a mixture containing 2 parts by weight of component X and 5 parts by weight component Y, X and Y are present at a weight ratio of 2:5, and are present in such ratio regardless of whether additional components are contained in the mixture.

A weight percent (wt. %) of a component, unless specifically stated to the contrary, is based on the total weight of the formulation or composition in which the component is included. As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

The term “aliphatic” as used herein refers to a non-aromatic hydrocarbon group and includes branched and unbranched, alkyl, alkenyl, or alkynyl groups.

The term “alkyl” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The symbols Aⁿ is used herein as merely a generic substituent in the definitions below.

The term “alkoxy” as used herein is an alkyl group bound through a single, terminal ether linkage; that is, an “alkoxy” group can be defined as —OA¹ where A¹ is alkyl as defined above.

The term “alkenyl” as used herein is a hydrocarbon group of from 2 to 24 carbon atoms with a structural formula containing at least one carbon-carbon double bond. Asymmetric structures such as (A¹A²)C=C(A³A⁴) are intended to include both the E and Z isomers. This may be presumed in structural formulae herein wherein an asymmetric alkene is present, or it may be explicitly indicated by the bond symbol C=C. The alkenyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “alkynyl” as used herein is a hydrocarbon group of 2 to 24 carbon atoms with a structural formula containing at least one carbon-carbon triple bond. The alkynyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “aryl” as used herein is a group that contains any carbon-based aromatic group including, but not limited to, benzene, naphthalene, phenyl, biphenyl, phenoxybenzene, and the like. The term “heteroaryl” is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is included in the term “aryl,” defines a group that contains an aromatic group that does not contain a heteroatom. The aryl and heteroaryl group can be substituted or unsubstituted. The aryl and heteroaryl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

The term “aldehyde” as used herein is represented by the formula —C(O)H. Throughout this specification “C(O)” is a shorthand notation for C═O.

The terms “amine” or “amino” as used herein are represented by the formula NA¹A²A³, where A¹, A², and A³ can be, independently, hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “carboxylic acid” as used herein is represented by the formula —C(O)OH. A “carboxylate” as used herein is represented by the formula —C(O)O—.

The term “ester” as used herein is represented by the formula —OC(O)A¹ or —C(O)OA¹, where A¹ can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula A¹OA², where A¹ and A² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula A¹C(O)A², where A¹ and A² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “halide” as used herein refers to the halogens fluorine, chlorine, bromine, and iodine.

The term “hydroxyl” as used herein is represented by the formula —OH.

The term “nitro” as used herein is represented by the formula —NO₂.

The term “cyano” as used herein is represented by the formula —CN

The term “azido” as used herein is represented by the formula —N₃.

The term “sulfonyl” is used herein to refer to the sulfo-oxo group represented by the formula —S(O)₂A¹, where A¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “sulfonylamino” or “sulfonamide” as used herein is represented by the formula —S(O)₂NH₂.

The term “thiol” as used herein is represented by the formula —SH.

It is to be understood that the compounds provided herein may contain chiral centers. Such chiral centers may be of either the (R-) or (S-) configuration. The compounds provided herein may either be enantiomerically pure or be diastereomeric or enantiomeric mixtures. It is to be understood that the chiral centers of the compounds provided herein may undergo epimerization in vivo. As such, one of skill in the art will recognize that administration of a compound in its (R-) form is equivalent, for compounds that undergo epimerization in vivo, to administration of the compound in its (S-) form.

As used herein, substantially pure means sufficiently homogeneous to appear free of readily detectable impurities as determined by standard methods of analysis, such as thin layer chromatography (TLC), nuclear magnetic resonance (NMR), gel electrophoresis, high performance liquid chromatography (HPLC) and mass spectrometry (MS), gas-chromatography mass spectrometry (GC-MS), and similar, used by those of skill in the art to assess such purity, or sufficiently pure such that further purification would not detectably alter the physical and chemical properties, such as enzymatic and biological activities, of the substance. Both traditional and modern methods for purification of the compounds to produce substantially chemically pure compounds are known to those of skill in the art. A substantially chemically pure compound may, however, be a mixture of stereoisomers. Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

A “pharmaceutically acceptable” component is one that is suitable for use with humans and/or animals without undue adverse side effects (such as toxicity, irritation, and allergic response) commensurate with a reasonable benefit/risk ratio.

“Pharmaceutically acceptable salt” refers to a salt that is pharmaceutically acceptable and has the desired pharmacological properties. Such salts include those that may be formed where acidic protons present in the compounds are capable of reacting with inorganic or organic bases. Suitable inorganic salts include those formed with the alkali metals, e.g., sodium, potassium, magnesium, calcium, and aluminum. Suitable organic salts include those formed with organic bases such as the amine bases, e.g., ethanolamine, diethanolamine, triethanolamine, tromethamine, N-methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). When two acidic groups are present, a pharmaceutically acceptable salt may be a mono-acid-mono-salt or a di-salt; similarly, where there are more than two acidic groups present, some or all of such groups can be converted into salts.

“Pharmaceutically acceptable excipient” refers to an excipient that is conventionally useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

A “pharmaceutically acceptable carrier” is a carrier, such as a solvent, suspending agent or vehicle, for delivering the disclosed compounds to the patient. The carrier can be liquid or solid and is selected with the planned manner of administration in mind. Liposomes are also a pharmaceutical carrier. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated.

The term “therapeutically effective amount” as used herein means that amount of active compound or pharmaceutical agent that elicits the biological or medicinal response in a tissue, system, animal or human that is being sought by a researcher, veterinarian, medical doctor or other clinician. In reference to cancers or other unwanted cell proliferation, an effective amount comprises an amount sufficient to cause a tumor to shrink and/or to decrease the growth rate of the tumor (such as to suppress tumor growth) or to prevent or delay other unwanted cell proliferation. In some embodiments, an effective amount is an amount sufficient to delay development. In some embodiments, an effective amount is an amount sufficient to prevent or delay occurrence and/or recurrence. An effective amount can be administered in one or more doses. In the case of cancer, the effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer.

Effective amounts of a compound or composition described herein for treating a mammalian subject can include about 0.1 to about 1000 mg/Kg of body weight of the subject/day, such as from about 1 to about 100 mg/Kg/day, especially from about 10 to about 100 mg/Kg/day. The doses can be acute or chronic. A broad range of disclosed composition dosages are believed to be both safe and effective.

Reference will now be made in detail to specific aspects of the disclosed materials, compounds, compositions, articles, and methods, examples of which are illustrated in the accompanying Examples and Figures.

### Processes for Synthesis of Withanolide D and Withanolide A

The present disclosure provides processes for the synthesis of Withanolide D and Withanolide A. The present disclosure also provides these compounds prepared by the discloses processes.

In one aspect, a process is provided for synthesizing Withanolide D having the structure

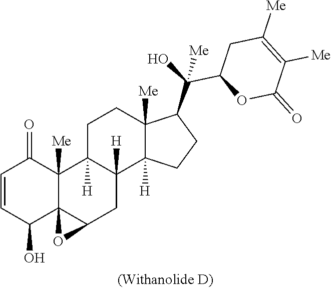

comprising treating a compound of Formula I

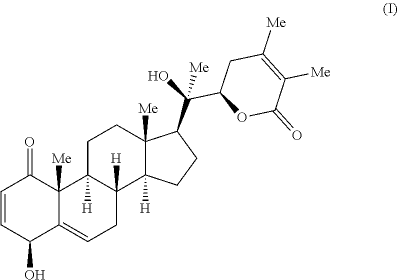

with a titanium alkoxide and tert-butyl hydroperoxide to provide Withanolide D. In some aspects, the titanium alkoxide comprises titanium isopropoxide. In some aspects, the above process is performed in the presence of a solvent. The solvent may typically comprise one or more solvents which are unreactive with the components of the described process. In some aspects, the solvent comprises chloroform. Other suitable solvents or combinations thereof may be used as would be apparent to a person of skill in the art. In some aspects, the process is performed at a temperature of about −30 degrees Celsius.

In another aspect, the above process further comprises preparing the compound of Formula I by a process comprising treating a compound of Formula II

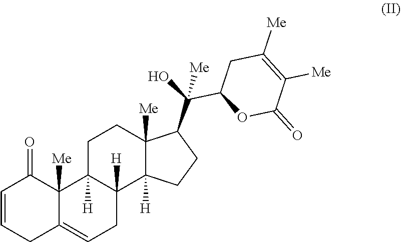

with selenium dioxide to provide the compound of Formula I.

In some aspects, the above process is performed in the presence of a solvent. The solvent may typically comprise one or more solvents which are unreactive with the components of the described process. In some aspects, the solvent comprises an ethereal solvent, for example, diethyl ether, tetrahydrofuran, 1,4 dioxane, or dimethoxyethane. Other suitable solvents or combinations may be used as would be apparent to a person of skill in the art. In some embodiments, the above process is performed at room temperature.
In another aspect, a process is provided for synthesizing Withanolide A having the structure

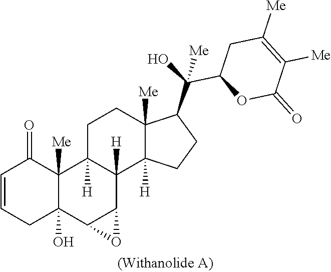

comprising treating Withacoagin having the structure

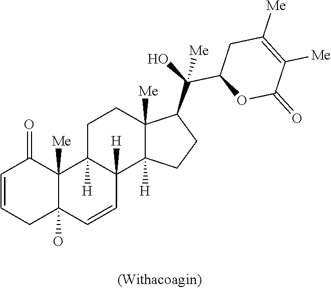

with a titanium alkoxide and tert-butyl hydroperoxide to provide Withanolide A. In some aspects, the titanium alkoxide comprises titanium isopropoxide. In some aspects, the above process is performed in the presence of a solvent. The solvent may typically comprise one or more solvents which are unreactive with the components of the described process. In some aspects, the solvent comprises chloroform. Other suitable solvents or combinations thereof may be used as would be apparent to a person of skill in the art. In some aspects, the above process is performed at a temperature of about −10 degrees Celsius.

In another aspect, the above process further comprises preparing Withacoagin by a process comprising treating a compound of Formula II

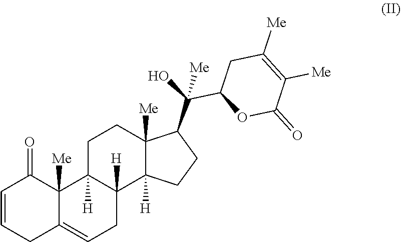

with light irradiation in the presence of a photocatalyst and oxygen followed by treatment with a reductant to provide Withacoagin.

In some aspects, the photocatalyst comprises 4,5,6,7-tetrachloro-2′,4′,5′,7′-tetraiodofluorescein. In some aspects, the reductant comprises triethyl phosphite. In some aspects, the above process is performed in the presence of a solvent. The solvent may typically comprise one or more solvents which are unreactive with the components of the described process. In some aspects, the solvent comprises pyridine. Other suitable solvents or combinations thereof may be used as would be apparent to a person of skill in the art. In some aspects, the light irradiation is provided by a suitable visible light source as would be apparent to a person of skill in the art (for example, a white light emitting diode).

In another aspect, the above processes further comprise preparing the compound of Formula II by a process comprising removing a protecting group PG from a compound of Formula III

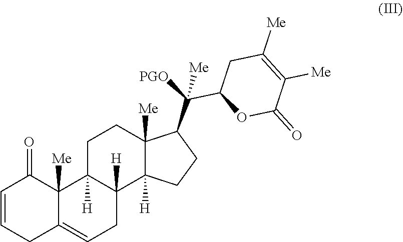

to provide a compound of Formula II.

The protecting group PG may comprise any suitable protecting group for an alcohol as would be apparent to a person of skill in the art. Suitable protecting groups include, but are not limited to, acetyl (Ac), benzoyl (Bz), benzyl (Bn), methoxyethoxymethyl (MEM), dimethoxytrityl (DMT), methoxymethyl (MOM), methoxytrityl (MMT), p-methoxybenzyl (PMB), p-methoxyphenyl (PMP), methylthiomethyl, pivaloyl, tert-butyl (tBu), tetrahydropyranyl (THP), tetrahydrofuran (THF), trityl (Tr), silyl ethers (such as trimethylsilyl (TMS), tert-butyldimethylsilyl (TBDMS or TBS), or triisopropylsilyl (TIPS)), methyl, and ethoxyethyl (EE). In particular aspects, the protecting group PG comprises methoxymethyl (MOM). In some aspects, the above process is performed in the presence of a solvent. The solvent may typically comprise one or more solvents which are unreactive with the components of the described process. Suitable solvents or combinations thereof which may be used as would be apparent to a person of skill in the art.

In another aspect, the above processes further comprise preparing the compound of Formula III by a process comprising treating a compound of Formula IV

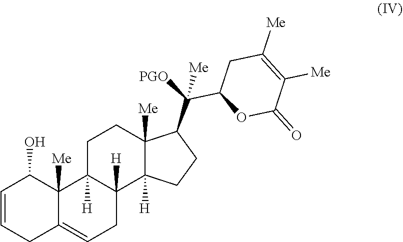

with an oxidant to provide the compound of Formula III.

In some aspects, the oxidant comprises 1,1,1-tris(acetyloxy)-1,1-dihydro-1,2-benziodoxol-3-(1H)-one, i.e., Dess Martin periodinane. In some aspects, the above process is performed in the presence of a base, for example, sodium bicarbonate. In some aspects, the above process is performed in the presence of a solvent. The solvent may typically comprise one or more solvents which are unreactive with the components of the described process. In some aspects, the solvent comprises dichloromethane. Other suitable solvents or combinations thereof may be used as would be apparent to a person of skill in the art. In some aspects, the above process is performed at about room temperature.

In some aspects, the above processes further comprise preparing the compound of Formula IV by a process comprising treating a compound of Formula V

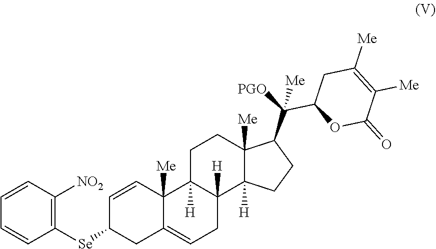

with an oxidant followed by a base to provide the compound of Formula IV.

In some aspects, the oxidant comprises meta-chloroperoxybenzoic acid, In some aspects, the base may comprise an alkoxide, for example sodium methoxide, optionally as a solution in methanol. In some aspects, the above process is performed in the presence of a solvent. The solvent may typically comprise one or more solvents which are unreactive with the components of the described process. In some aspects, the solvent comprises dichloromethane. Other suitable solvents or combinations thereof may be used as would be apparent to a person of skill in the art. In some aspects, the above process is performed at a temperature of about −78 degrees Celsius followed by warming to a range from about 0 degrees Celsius to room temperature.

In some aspects, the above processes further comprise preparing the compound of Formula V by a process comprising treating a compound of Formula VI

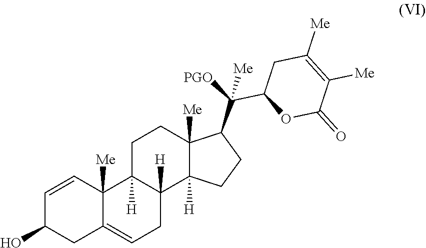

with 2-nitrophenyl selenocyanate in the presence of a phosphine to provide the compound of Formula V.

In some aspects, the phosphine comprises a trialkyl phosphine or a triaryl phosphine, for example tributyl phosphine. In some aspects, the above process is performed in the presence of a solvent. The solvent may typically comprise one or more solvents which are unreactive with the components of the described process. In some aspects, the solvent comprises an ethereal solvent, for example diethyl ether, tetrahydrofuran, 1,4-dioxane, or dimethoxyethane. Other suitable solvents or combinations thereof may be used as would be apparent to a person of skill in the art. In some aspects, the above process is performed at room temperature.

In some aspects, the above processes further comprise preparing the compound of Formula VI by a process comprising treating a compound of Formula VII

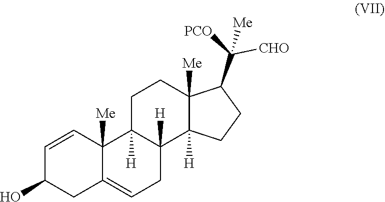

with a compound of Formula VIII

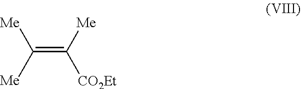

which has been previously treated with an amide base to provide the compound of Formula VI.

In some aspects, the amide base comprises lithium bis(trimethylsilyl)amide, sodium bis(trimethylsilyl)amide, or lithium diisopropylamide. In some aspects, the above process is performed in the presence of a solvent. The solvent may typically comprise one or more solvents which are unreactive with the components of the described process. In some aspects, the solvent comprises an ethereal solvent, for example diethyl ether, tetrahydrofuran, 1,4-dioxane, or dimethoxyethane. In some aspects, the solvent may further comprise a polar aprotic solvent such as N,N′-dimethylpropyleneurea (DMPU) or hexamethylphosphoramide (HMPA). Other suitable solvents or combinations thereof may be used as would be apparent to a person of skill in the art. In some aspects, the above process is performed at a temperature of about −78 degrees Celsius to about −40 degrees Celsius.

In some aspects, the above processes further comprise preparing the compound of Formula VII by a process comprising treating a compound of Formula IX

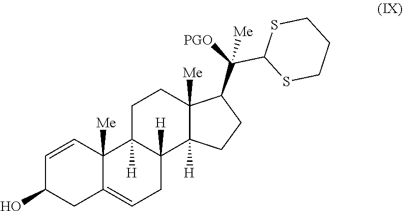

under reaction conditions suitable for conversion of the 1,3-dithiolane moiety in Formula IX into an aldehyde moiety to provide the compound of Formula VII.

Suitable conditions for conversion of the 1,3-dithiolane moiety into an aldehyde moiety will be apparent to those skilled in the art. Representative examples include, but are not limited to, Brønsted acids, Lewis acids, and oxidants. In some embodiments, the 1,3-dithiolane moiety is converted to an aldehyde moiety by treatment with N-bromosuccinimide. In some aspects, the above process is performed in the presence of a solvent. The solvent may typically comprise one or more solvents which are unreactive with the components of the described process. Other suitable solvents or combinations thereof may be used as would be apparent to a person of skill in the art.

In some aspects, the above processes further comprise preparing the compound of Formula IX by a process comprising treating a compound of Formula X

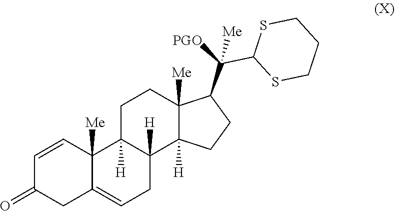

with sodium borohydride in the presence of cerium trichloride to provide the compound of Formula IX.

In some aspects, the above process is performed in the presence of a solvent. The solvent may typically comprise one or more solvents which are unreactive with the components of the described process. In some aspects, the solvent comprises dichoromethane and methanol. Other suitable solvents or combinations thereof may be used as would be apparent to a person of skill in the art. In some aspects, the above process is performed at a temperature of about 0 degrees Celsius.

In some aspects, the above processes further comprise preparing the compound of Formula X by a process comprising treating a compound of Formula XT

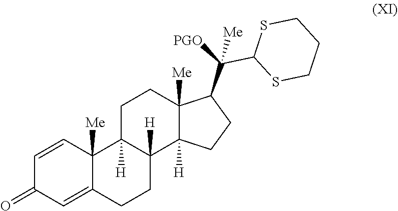

with an alkoxide base to provide the compound of Formula X.

In some aspects, the alkoxide base comprises potassium tert-butoxide. In some aspects, the above process is performed in the presence of a solvent. The solvent may typically comprise one or more solvents which are unreactive with the components of the described process. In some aspects, the solvent comprises an ethereal solvent, for example diethyl ether, tetrahydrofuran, 1,4-dioxane, or dimethoxyethane. Other suitable solvents or combinations thereof may be used as would be apparent to a person of skill in the art.

In some aspects, the above processes further comprise preparing the compound of Formula XI by a process comprising treating a compound of Formula XII

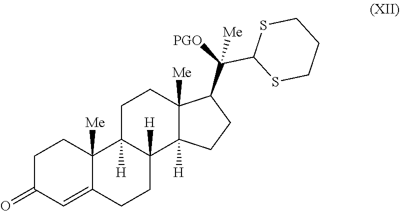

with an amide base followed by treating with N-tert-butyl phenylsulfinimidoyl chloride (i.e., the Mukaiyama reagent) to provide the compound of Formula XI.

In some embodiments, the amide base may comprise lithium bis(trimethylsilyl)amide, sodium bis(trimethylsilyl)amide, or lithium diisopropylamide. In some aspects, the above process is performed in the presence of a solvent. The solvent may typically comprise one or more solvents which are unreactive with the components of the described process. In some aspects, the solvent comprises an ethereal solvent, for example diethyl ether, tetrahydrofuran, 1,4-dioxane, or dimethoxyethane. Other suitable solvents or combinations thereof may be used as would be apparent to a person of skill in the art. In some aspects, the above process is performed at a temperature of about −78 degrees Celsius to about −40 degrees Celsius.

In some aspects, the above processes further comprise preparing the compound of Formula XII by a process comprising treating a compound of Formula XIII

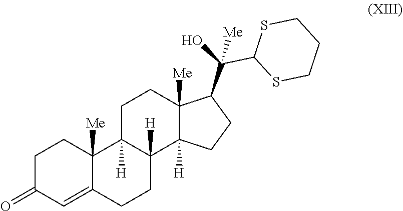

under suitable reaction conditions for introduction of a protection group PG to provide the compound of Formula XII.

The protecting group PG may comprise any suitable protecting group for an alcohol as would be apparent to a person of skill in the art. Suitable protecting groups include, but are not limited to, acetyl (Ac), benzoyl (Bz), benzyl (Bn), methoxyethoxymethyl (MEM), dimethoxytrityl (DMT), methoxymethyl (MOM), methoxytrityl (MMT), p-methoxybenzyl (PMB), p-methoxyphenyl (PMP), methylthiomethyl, pivaloyl, tert-butyl (tBu), tetrahydropyranyl (THP), tetrahydrofuran (THF), trityl (Tr), silyl ethers (such as trimethylsilyl (TMS), tert-butyldimethylsilyl (TBDMS or TBS), or triisopropylsilyl (TIPS)), methyl, and ethoxyethyl (EE). In particular aspects, the protecting group PG comprises methoxymethyl (MOM). In some aspects, the above process is performed in the presence of a solvent. The solvent may typically comprise one or more solvents which are unreactive with the components of the described process. Other suitable solvents or combinations thereof may be used as would be apparent to a person of skill in the art.

In some aspects, the above processes further comprise preparing the compound of Formula XIII by a process comprising treating a compound of Formula XIV

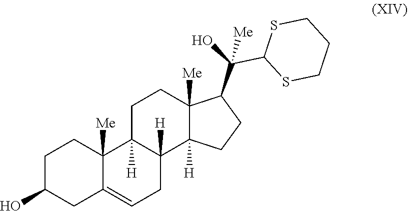

with aluminum isopropoxide to provide the compound of Formula XIII. In some aspects, the above process may further comprise a ketone reagent, for example N-methyl-4-piperidone. In some aspects, the above process is performed in the presence of a solvent. The solvent may typically comprise one or more solvents which are unreactive with the components of the described process. In some aspects, the solvent comprises toluene. Other suitable solvents or combinations thereof may be used as would be apparent to a person of skill in the art. In some aspects, the above process is performed at an approximate temperature in which the solvent is at reflux.

In some aspects, the above processes further comprise preparing the compound of Formula XIV by a process comprising treating Pregnenolone

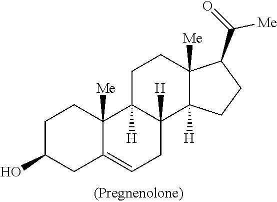

with 1,3-dithiane that has been previously treated with an organolithium base to provide the compound of Formula XIV.

In some aspects, the organolithium base comprises methyllithium, n-butyllithium, sec-butyllithium, isopropyllithium, tert-butyllithium, or phenyllithium. In some aspects, the above process is performed in the presence of a solvent. The solvent may typically comprise one or more solvents which are unreactive with the components of the described process. In some aspects, the solvent comprises an ethereal solvent, for example diethyl ether, tetrahydrofuran, 1,4-dioxane, or dimethoxyethane. Other suitable solvents or combinations thereof may be used as would be apparent to a person of skill in the art.

In another aspect, Withanolide D is provided prepared by a process described herein.

In another aspect, Withanolide A is provided prepared by a process described herein.

In another aspect, a process is provided of preparing a compound of Formula A

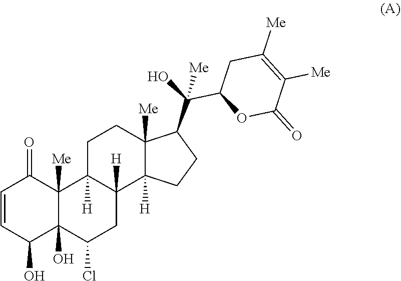

comprising treating Withanolide D

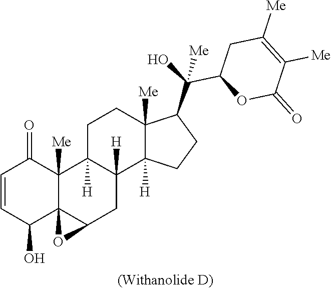

with cerium trichloride to provide the compound of Formula A.

In some aspects, the above process is performed in the presence of a solvent. The solvent may typically comprise one or more solvents which are unreactive with the components of the described process. In some aspects, the solvent comprises acetonitrile, an ethereal solvent (such as tetrahydrofuran), or combinations thereof. Other suitable solvents or combinations thereof may be used as would be apparent to a person of skill in the art.

In another aspect, a process is provided of preparing a compound of Formula B

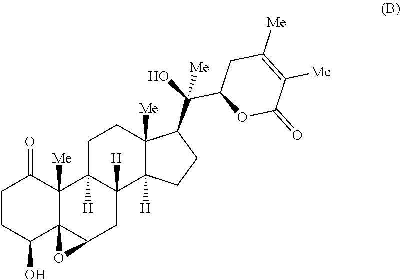

comprising treating Withanolide D

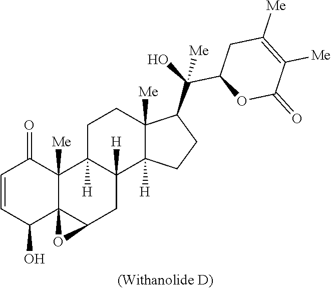

under hydrogenation conditions to provide the compound of Formula B.

In some aspects, the hydrogenation conditions comprise treating with hydrogen and a catalyst (for example a palladium or platinum catalyst, such as palladium on carbon). In some aspects, the above process is performed in the presence of a solvent. The solvent may typically comprise one or more solvents which are unreactive with the components of the described process. Other suitable solvents or combinations thereof may be used as would be apparent to a person of skill in the art.

Variations on compounds made by or used in the processes described herein can include the addition, subtraction, or movement of the various constituents as described for each compound. Similarly, when one or more chiral centers is present in a molecule the chirality of the molecule can be changed. Additionally, the synthesis of the compounds for use in the process can involve the protection of various chemical groups, and further the compounds prepared by the disclosed processes may be subsequently deprotected as appropriate. The use of protection and deprotection, and the selection of appropriate protecting groups would be readily known to one skilled in the art. The chemistry of protecting groups can be found, for example, in Peter G. M. Wuts, Greene's Protective Groups in Organic Synthesis, 5^{th} Ed., Wiley & Sons, 2014.

The described processes, or reactions to produce the compounds used in the described processes, can be carried out in solvents indicated herein, or in solvents which can be selected by one of skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry, or by chromatography such as high-performance liquid chromatography (HPLC) or thin layer chromatography.

### Other Aspects of Disclosure

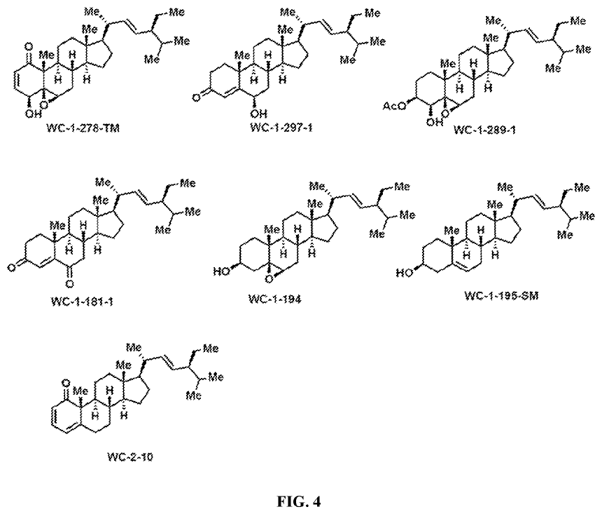

Also disclosed herein are withaferin analogs of Formula C:

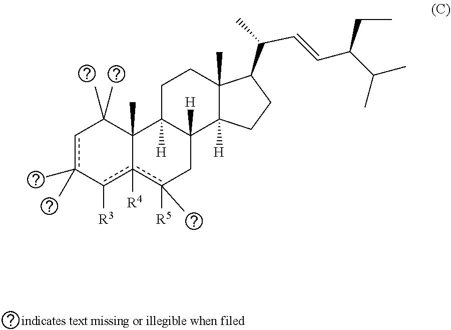

wherein the dashed line ---- represents a bond that is present or absent,
R¹ and R^{1′} are independently chosen from H, OH, and OAc, or R¹ and R^{1′}, together are O;
R² and R^{2′} are independently chosen from null, H, OH, and OAc, or R² and R^{2′} together are O;
R³ is chosen from H or OH;
R⁴ is chosen from null, or R⁴ and R⁵ together are O;
R⁵ and R^{5′} are independently chose from null, H, and OH, or R⁵ and R^{5′} together are O, or
R⁵ and R⁴ together are O.
In some examples, disclosed herein are compounds as shown in FIG. 4.

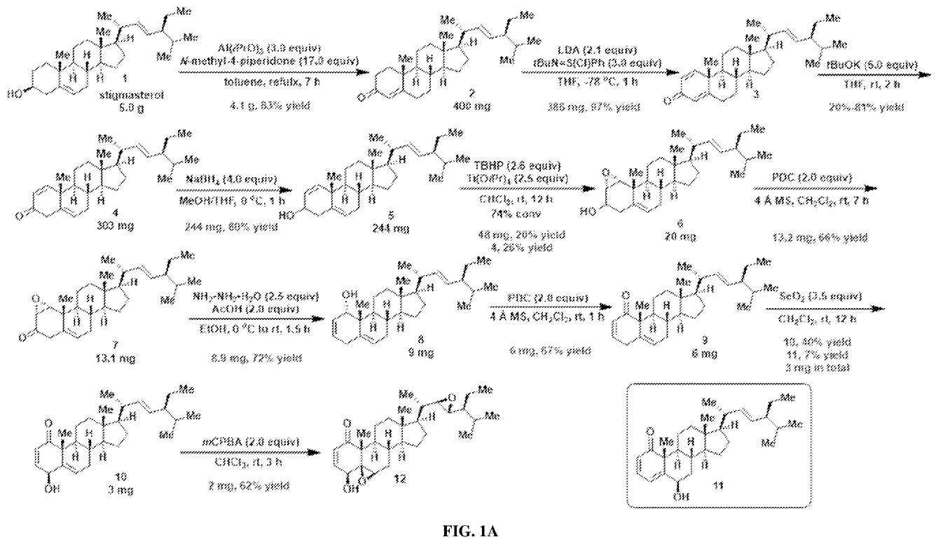

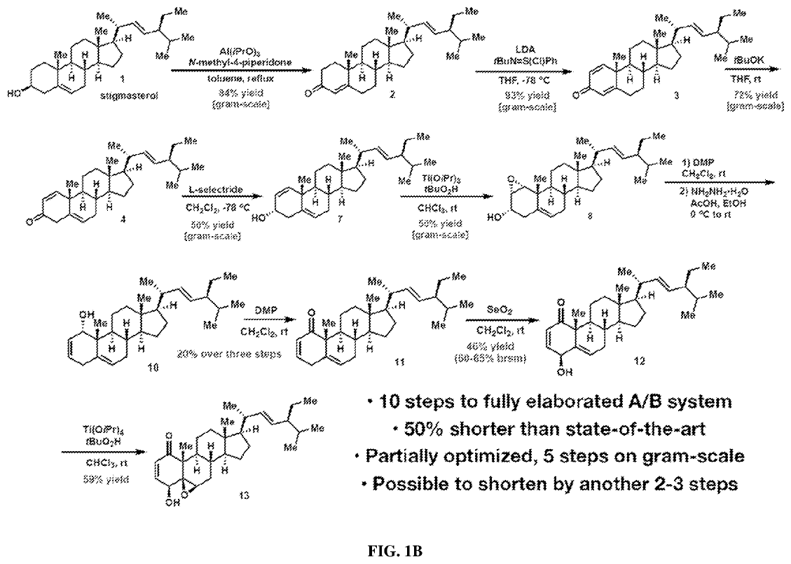

Disclosed herein is a method of synthesizing withanolides such as withaferin and withacnistin. The synthetic routes are illustrated in FIGS. 1A and 1B.

Step 1 of the synthesis begins by oxidizing stigmasterol. Suitable oxidizing agents include aluminum isopropoxide (Oppenauer oxidation), pyridinium dichromate, pyridinium chlorochromate, chromic acid/pyridine, manganese dioxide, or Swern reagents (DMSO, oxalyl chloride, and amine).

Step 2 involves dehydrogenation of the oxidized stigmasterol with to produce the diene-one derivative. Suitable reagents include lithium diisopropylamide (LDA), lithium aluminum hydroxide (LAH), and diisobutyl aluminum hydride.

Step 3 involves the isomerization of the double bond across the fused ring system with base such as potassium tert-butoxide or sodium hydride in hexamethylphosphoramide.

Step 4 involves reducing the ketone to an alcohol with a reducing agent. Suitable reducing agents include sodium borohydride, lithium aluminum hydride, L-selectride.

Step 5 involves epoxidation with tert-butyl hydroperoxide, titanium isopropoxide, or other suitable epoxidation reagent.

Step 6 involves reoxidizing the alcohol to a ketone with an oxidation reagent.

Suitable oxidizing agents include aluminum isopropoxide (Oppenauer oxidation), pyridinium dichromate, pyridinium chlorochromate, chromic acid/pyridine, manganese dioxide, or Swern reagents (DMSO, oxalyl chloride, and amine).

Step 7 involves conversion of the keto-epoxide into an enol with ammonium followed by acid (e.g., acetic acid).

Step 8 involves oxidizing the enol into an enone with an oxidation reagent. Suitable oxidizing agents include aluminum isopropoxide (Oppenauer oxidation), pyridinium dichromate, pyridinium chlorochromate, chromic acid/pyridine, manganese dioxide, or Swern reagents (DMSO, oxalyl chloride, and amine).

Step 9 involves hydroxylation of the enone with selenium oxide.

Step 10 involves the double epoxidation with meta-chloroperoxybenzoic acid, titanium isopropoxide and the like.

In some aspects, the following particular embodiments of the disclosure are also provided:

Embodiment 1. A process for synthesizing Withanolide D having the structure

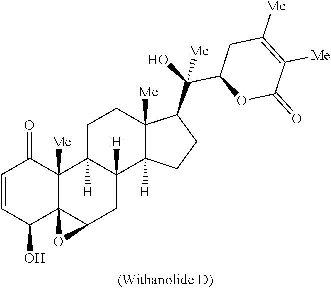

comprising treating a compound of Formula I

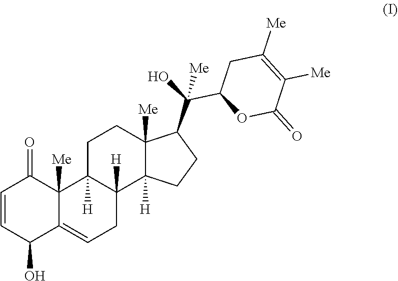

with a titanium alkoxide (such as titanium isopropoxide) and tert-butyl hydroperoxide to provide Withanolide D.

Embodiment 2. The process of embodiment 1, further comprising preparing the compound of Formula I by a process comprising treating a compound of Formula II

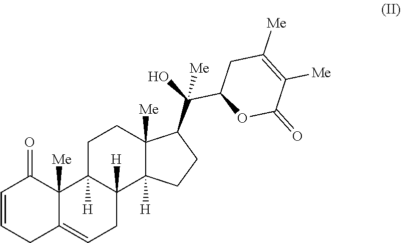

with selenium dioxide to provide the compound of Formula I.

Embodiment 3. A process for synthesizing Withanolide A having the structure

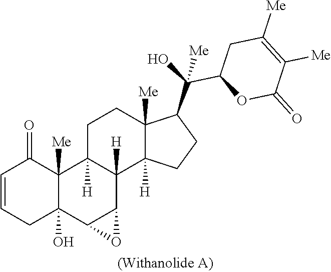

comprising treating Withacoagin having the structure

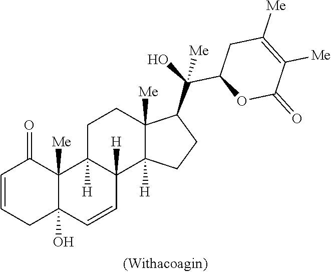

with a titanium alkoxide (such as titanium isopropoxide) and tert-butyl hydroperoxide to provide Withanolide A.
Embodiment 4. The process of embodiment 3, further comprising preparing Withacoagin by a process comprising treating a compound of Formula II

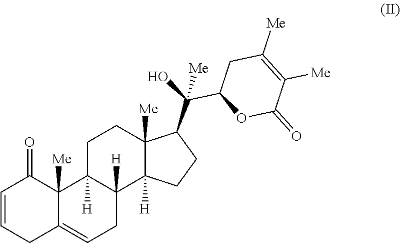

with light irradiation in the presence of a photocatalyst (such as 4,5,6,7-tetrachloro-2′,4′,5′,7′-tetraiodofluorescein) and oxygen followed by treatment with a reductant (such as triethyl phosphite) to provide Withacoagin.
Embodiment 5. The process of embodiment 2 or embodiment 4, further comprising preparing the compound of Formula II by a process comprising removing a protecting group PG (such as methoxymethyl) from a compound of Formula III

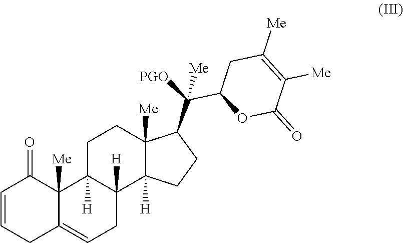

to provide a compound of Formula II.

Embodiment 6. The process of embodiment 5, further comprising preparing the compound of Formula III by a process comprising treating a compound of Formula IV

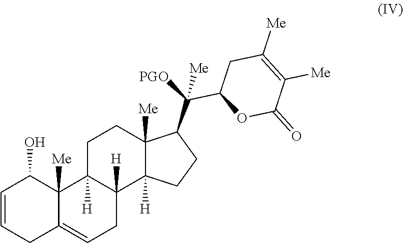

with an oxidant (such as 1,1,1-tris(acetyloxy)-1,1-dihydro-1,2-benziodoxol-3-(1H)-one) to provide the compound of Formula III.

Embodiment 7. The process of embodiment 6, further comprising preparing the compound of Formula IV by a process comprising treating a compound of Formula V

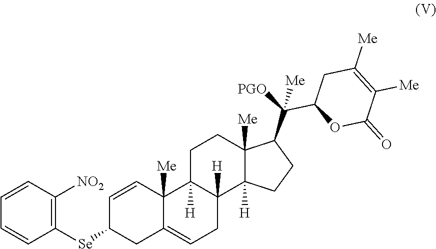

with an oxidant (such as meta-chloroperoxybenzoic acid) followed by a base (such as an alkoxide, for example sodium methoxide) to provide the compound of Formula IV.

Embodiment 8. The process of embodiment 7, further comprising preparing the compound of Formula V by a process comprising treating a compound of Formula VI

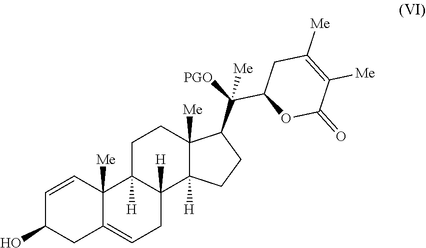

with 2-nitrophenyl selenocyanate in the presence of a phosphine (such as tributylphosphine) to provide the compound of Formula V.

Embodiment 9. The process of embodiment 8, further comprising preparing the compound of Formula VI by a process comprising treating a compound of Formula VII

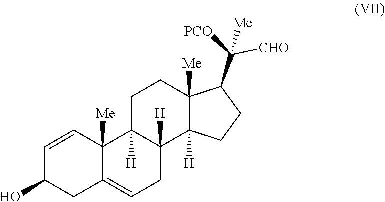

with a compound of Formula VIII

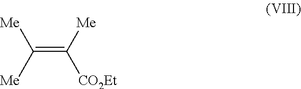

which has been previously treated with an amide base (such as lithium bis(trimethylsilyl)amide) to provide the compound of Formula VI.

Embodiment 10. The process of embodiment 9, further comprising preparing the compound of Formula VII by a process comprising treating a compound of Formula IX

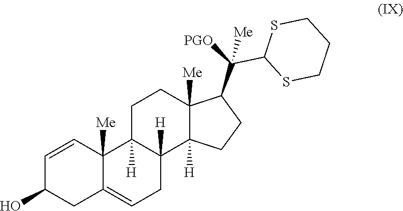

under reaction conditions suitable for conversion of the 1,3-dithiolane moiety in Formula IX into an aldehyde moiety (for example, by treating with N-bromosuccinimide) to provide the compound of Formula VII.

Embodiment 11. The process of embodiment 10, further comprising preparing the compound of Formula IX by a process comprising treating a compound of Formula X

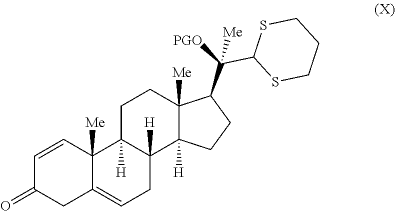

with sodium borohydride in the presence of cerium trichloride to provide the compound of Formula IX.

Embodiment 12. The process of embodiment 11, further comprising preparing the compound of Formula X by a process comprising treating a compound of Formula XI

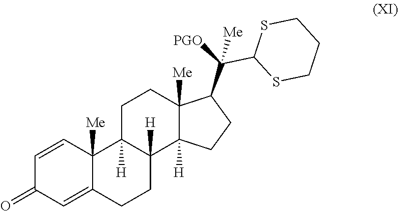

with an alkoxide base (such as potassium tert-butoxide) to provide the compound of Formula X.

Embodiment 13. The process of embodiment 12, further comprising preparing the compound of Formula XI by a process comprising treating a compound of Formula XII

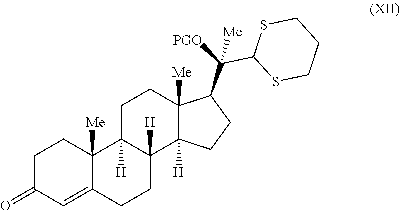

with an amide base (for example, lithium bis(trimethylsilyl)amide) followed by treating with N-tert-butyl phenylsulfinimidoyl chloride to provide the compound of Formula XI.

Embodiment 14. The process of embodiment 13, further comprising preparing the compound of Formula XII by a process comprising treating a compound of Formula XIII

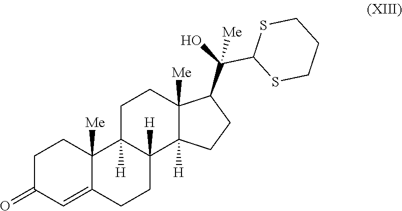

under suitable reaction conditions for introduction of a protection group PG (for example, by treatment with methoxymethyl chloride and a base when PG is methoxymethyl) to provide the compound of Formula XII.

Embodiment 15. The process of embodiment 14, further comprising preparing the compound of Formula XIII by a process comprising treating a compound of Formula XIV

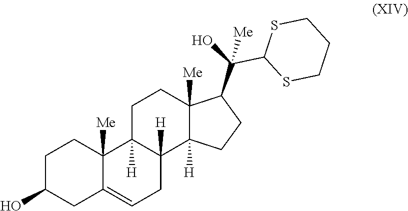

with aluminum isopropoxide to provide the compound of Formula XIII.

Embodiment 16. The process of embodiment 15, further comprising preparing the compound of Formula XIV by a process comprising treating Pregnenolone

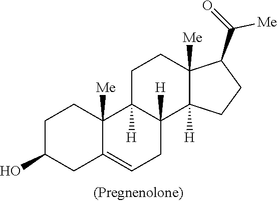

with 1,3-dithiane that has been previously treated with an organolithium base (such as n-butyl lithium) to provide the compound of Formula XIV.

Embodiment 17. Withanolide D prepared by a process of any of the proceeding embodiments:

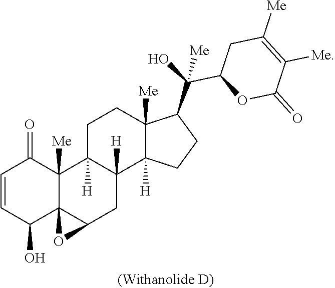

Embodiment 18. Withanolide A prepared by a process of any of the proceeding embodiments:

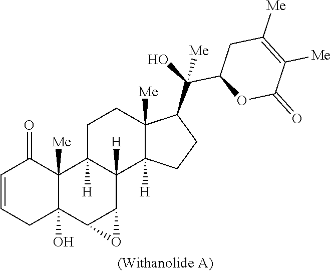

Embodiment 19. A process of preparing a compound of Formula A

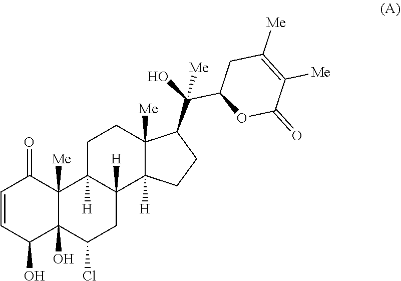

comprising treating Withanolide D

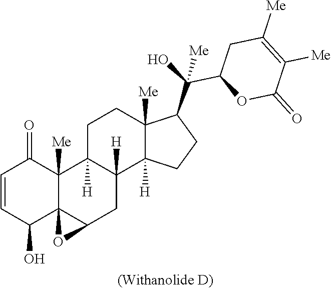

with cerium trichloride to provide the compound of Formula A.

Embodiment 20. A process of preparing a compound of Formula B

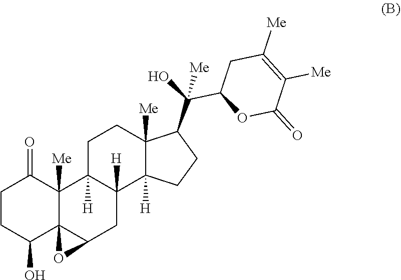

comprising treating Withanolide D

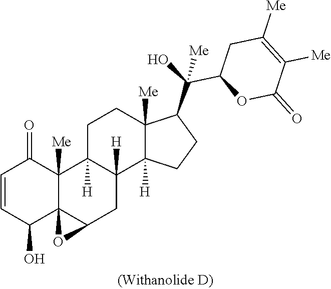

under hydrogenation conditions (for example, by treating with hydrogen in the presence of a palladium or platinum catalyst such as palladium on carbon) to provide the compound of Formula B.

Embodiment 21. A compound selected from:

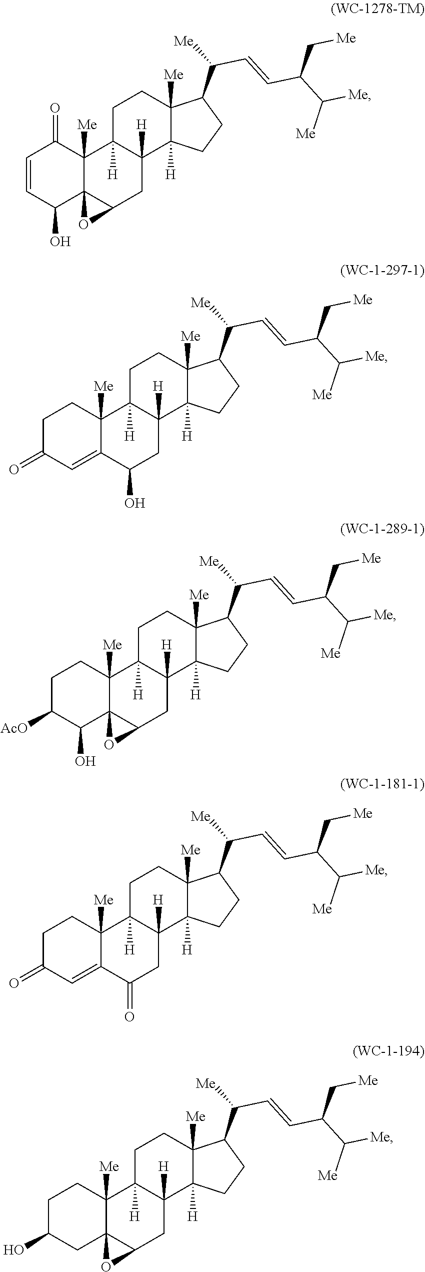

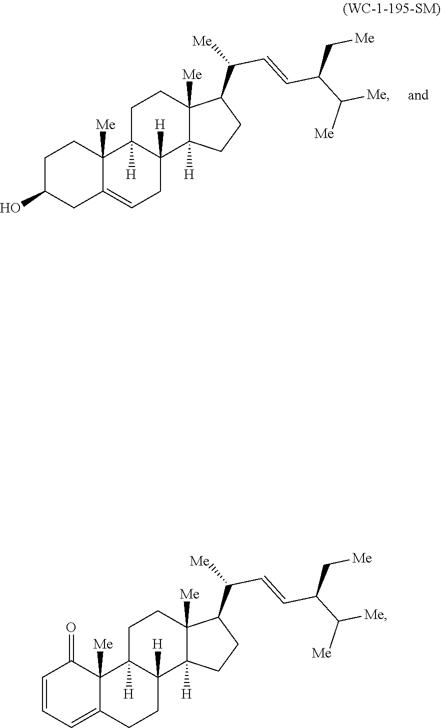

or a pharmaceutically acceptable salt or derivative thereof.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### Examples

The following examples are put forth to provide those of ordinary skill in the art with a complete disclosure and description of how the compounds, compositions, and methods claimed herein are made and evaluated and are intended to be purely exemplary of the invention and are not intended to limit the scope of what the inventors regard as their invention. Efforts have been made to ensure accuracy concerning numbers (e.g., amounts, temperature, etc.), but some errors and deviations should be accounted for. Unless indicated otherwise, parts are parts by weight, temperature is in degrees Celsius or is at ambient temperature, and pressure is at or near atmospheric pressure.

### Synthesis of Withaferin A and Withaferin D

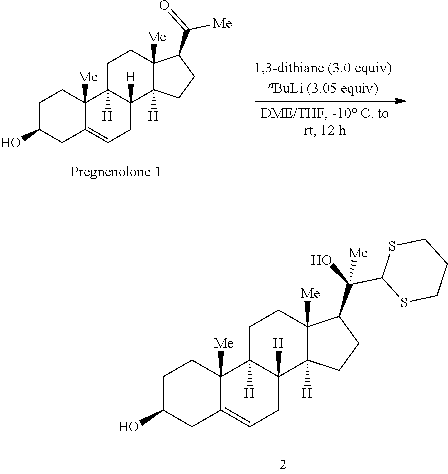

To a 1 L three-neck round bottom flask equipped with a stir bar and an addition funnel was added 1,3-dithane (11.4 g, 94.8 mmol, 3.0 equiv). The system was evacuated and charged with argon three times. Then to the cold solution of 1,3-dithane in THF (250 ML) at −10° C. was added ⁿBuLi (1.66 M, 58 mL, 96.4 mmol, 3.05 equiv) dropwise through the addition funnel. The solution was stirred at −10° C. for 2 h. Pregnenolone (10 g, 32 mmol, 1.0 equiv) was dissolved in dry THF (200 mL) using a 500 mL round bottom flask. Transferred the pregnenolone solution into the addition funnel via cannula. Kept the three-neck round bottom flask at −10° C., added the pregnenolone solution into the system and the resulting mixture was allowed to warm to RT slowly and was stirred overnight. The reaction was quenched with saturated NH₄Cl solution, then extracted with CH₂Cl₂, the combined organic phase was washed with H₂O, dried over Na₂SO₄ and concentrated to afford solid which was subjected to column flash chromatography (hex/EA=5:1 to 3:1) to afford dithiane 2 (13.1 g, 94% yield).

Physical State: white powder; R_{f}=0.2 (5:1 hexane: EA); [α]_{D}^{22.48}-57.3 (c 1.0, CHCl₃); ¹H NMR (500 MHz, Chloroform-d): δ 5.28 (d, J=5.1 Hz, 1H), 4.85 (d, J=7.2 Hz, 1H), 4.68 (d, J=7.2 Hz, 1H), 4.21 (s, 1H), 3.45 (tt, J=10.4, 4.3 Hz, 1H), 3.36 (s, 3H), 2.91-2.66 (m, 4H), 2.20 (m, 3H), 2.07-1.87 (m, 4H), 1.84-1.76 (m, 5H), 1.58 (dd, J=14.3, 6.8 Hz, 1H), 1.51 (s, 3H), 1.47-1.39 (m, 5H), 1.21 (td, J=12.5, 4.8 Hz, 1H), 1.13-0.96 (m, 3H), 0.94 (s, 3H), 0.86 (td, J=11.4, 5.4 Hz, 1H), 0.81 (s, 3H); ¹³C NMR (126 MHz, CDCl₃): δ 140.78, 121.51, 92.19, 81.79, 71.53, 61.66, 56.68, 56.60, 56.48, 49.92, 43.06, 42.21, 40.06, 37.22, 36.43, 32.29, 31.69, 31.66, 31.56, 31.34, 26.42, 23.93, 22.39, 21.84, 20.95, 19.34, 13.36; HRMS (ESI-TOF): Calcd for C₂₇H₄₄NaO₃S₂ ([M+Na]⁺): 503.2624; Found: 503.2614

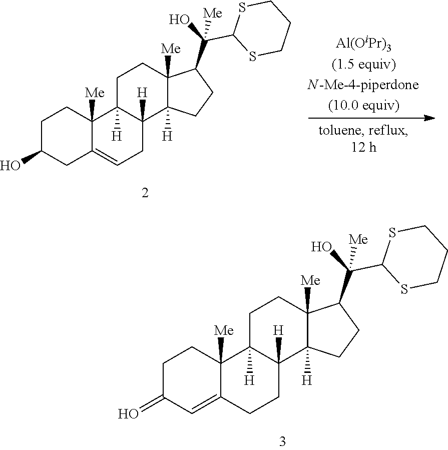

Dithiane 2 and Al(i-PrO)₃ (3.5. g, 17.2 mmol, 1.5 equiv) were added to a 250 mL three-neck round bottom flask. Dissolved the mixture with toluene (50 mL). To the stirred mixture was added N-methyl-4-piperidone (13.3 mL, 114.6 mmol, 10.0 equiv). The resulting mixture was refluxed for 12 h, then cooled, quenched the reaction with Rochelle's salt solution. Stirred it at room temperature for 1 h, then extracted with CH₂Cl₂. Collected the organic phase, washed with saturated NaCl, dried over Na₂SO₄, evaporated, and chromatographed on silica gel. Elution with CH₂Cl₂/MeOH (10:1 to 5:1) gave 2 (4.9 g, 98%) as white solid.

Physical State: white solid; R_{f}=0.3 (5:1 hexane: EA); [α]_{D}^{23.05}+33.9 (c 1.0, CHCl₃); ¹H NMR (500 MHz, Chloroform-d): δ 5.68 (s, 1H), 4.88 (d, J=7.1 Hz, 1H), 4.70 (d, J=7.1 Hz, 1H), 4.22 (s, 1H), 3.38 (s, 3H), 2.93-2.70 (m, 4H), 2.43-2.18 (m, 3H), 2.09-1.92 (m, 4H), 1.91-1.72 (m, 4H), 1.63 (tt, J=14.5, 6.7 Hz, 2H), 1.56-1.45 (m, 5H), 1.40 (qd, J=13.6, 13.2, 4.1 Hz, 1H), 1.24 (td, J=12.7, 4.5 Hz, 1H), 1.14 (s, 4H), 1.05-0.78 (m, 7H). ¹³C NMR (126 MHz, CDCl₃): δ 199.57, 171.53, 123.81, 92.31, 81.73, 61.68, 56.69, 55.69, 53.66, 43.19, 39.99, 38.57, 35.69, 35.05, 33.99, 32.93, 32.36, 31.88, 31.75, 26.47, 23.90, 22.40, 21.96, 20.95, 17.38. HRMS (ESI-TOF): Calcd for C₂₇H₄₃O₃S₂ ([M+H]⁺): 479.2648; Found: 479.2635.

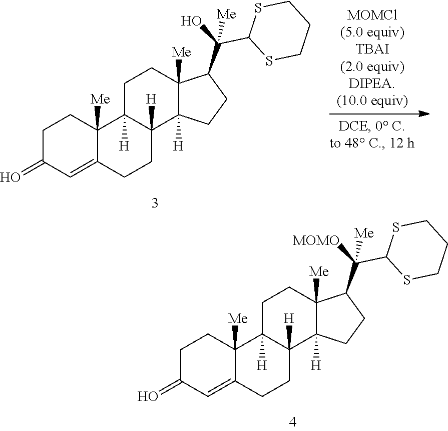

To a cold (0° C.) solution of compound 2 (8.2 g, 18.8. mmol, 1.0 equiv), TBAI (13.9 g, 37.6 mmol, 2.0 equiv) and DIPEA (31.1 mL, 188 mmol, 10.0 equiv) in dry DCE (150 mL) was added MOMCl (7.1 mL, 94 mmol, 5.0 equiv) dropwise. The mixture was allowed to warm to room temperature and stirred for 10 min and then heated to 48° C. for 12 h. Monitored the reaction by TLC until completion. The reaction mixture was allowed to cool to room temperature and quenched with saturated NaHCO₃ solution, and then extracted with CH₂Cl₂. The combined organic layers were washed with saturated NaCl, dried over Na₂SO₄, filtered and concentrated. The crude material was subjected to column chromatography on silica gel (hexane:EtOAc=5:1 to 3:1) to afford compound 4 (5.3 g, 52% yield) as a slightly yellowish solid.

Physical State: yellowish solid; R_{f}=0.41 (3:1 hexane: EA); [α]_{D}^{24.5}+64.8 (c 1.0, CHCl₃); ¹H NMR (500 MHz, Chloroform-d): δ 5.68 (s, 1H), 4.88 (d, J=7.1 Hz, 1H), 4.70 (d, J=7.1 Hz, 1H), 4.22 (s, 1H), 3.38 (s, 3H), 2.93-2.70 (m, 4H), 2.43-2.18 (m, 3H), 2.09-1.92 (m, 4H), 1.91-1.72 (m, 4H), 1.63 (u, J=14.5, 6.7 Hz, 2H), 1.56-1.45 (m, 5H), 1.40 (qd, J=13.6, 13.2, 4.1 Hz, 1H), 1.24 (td, J=12.7, 4.5 Hz, 1H), 1.14 (s, 4H), 1.05-0.78 (m, 7H). ¹³C NMR (126 MHz, CDCl₃): δ 199.57, 171.53, 123.81, 92.31, 81.73, 61.68, 56.69, 55.69, 53.66, 43.19, 39.99, 38.57, 35.69, 35.05, 33.99, 32.93, 32.36, 31.88, 31.75, 26.47, 23.90, 22.40, 21.96, 20.95, 17.38. HRMS (ESI-TOF): Calcd for C₂₇H₄₃O₃S₂ ([M+H]⁺): 479.2648; Found: 479.2635.

### Preparation of Mukaiyama Reagent:

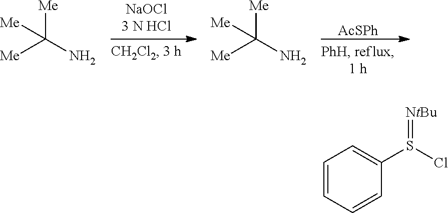

A suspension of tBuNH₂ (10 g, 14.4 mL, 136.7 mmol) and NaOCl (15% chlorine, 155 mL) in 360 mL CH₂Cl₂ was stirred and cooled in an ice-bath at 0° C. as 3N HCl (360 mL) was added dropwise during 1 h. Stirring and cooling were continued for additional 2 h. The layers were separated and the aqueous phase was extracted with CH₂Cl₂. Organic phase was combined and washed with saturated NaHCO₃, H₂O and saturated NaCl, dried over Na₂SO₄, filtered, and concentrated on a rotatory evaporator (the pressure should be >20 kPa, the temperature should be kept at 30° C.) to afford a pale-yellow liquid. The crude product (15.5 g, 80% crude yield) was highly volatile and used directly in the next step. The above procedure was repeated four times and 47.6 g product was combined.

A mixture of phenyl thioacetate (37.8 mL, 279 mmol) and N,N-dichloro-t-butylamine (47.6 g, 334.8 mmol) in 100 mL benzene was refluxed for 1 h. Volatile material was removed under reduced pressure and by azeotropic distillation on a rotary evaporator with benzene (4×30 mL) to give the reagent as a red-orange oil (63.2 g). The reagent partially solidified to yellow solid by keeping it still or by cooling it at 0° C. The crude product was used for the next step without further purification.

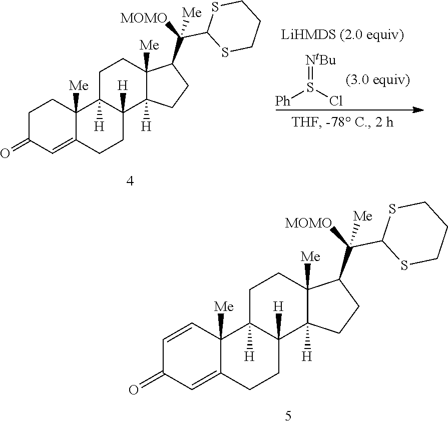

To a stirred solution of HMDS (44 mL, 209.9 mmol, 2.05 equiv) in 200 mL THF at 0° C. was added nBuLi (2.2 M in hexane, 93.1 mL, 204.8 mmol, 2.0 equiv) dropwise. The resulting mixture was stirred at 0° C. for 30 min. The freshly prepared LiHMDS solution was cooled to −78° C. before it was added enone 4 (49 g, 102.4 mmol, 1.0 equiv) in 300 mL dry THF. The resulting mixture was stirred at −78° C. for 30 min before it was added the freshly prepared N-tert-butyl phenylsulfinimidoyl chloride (66.3 g, 307.2 mmol, 3.0 equiv). The resulting mixture was stirred at −78° C. for an additional 2 h before it was quenched with sat. NH₄Cl solution. The layers were separated, and the aqueous layer was extracted with CH₂Cl₂ three times. The combined organic layers were washed with brine, dried with Na₂SO₄, filtered, and concentrated in vacuo. Flash column chromatography (silica gel, hexanes: EtOAc=10:1 to 5:1 to 3:1) afforded compound 5 (42.3 g, 86.6% yield).

Physical State: dark solid. The product can be recrystallized in Et₂O and CH₂Cl₂, and it would be very good white crystal. R_{f}=0.38 (2:1 hexane: EA); ¹H NMR (500 MHz, Chloroform-d): δ 6.99 (d, J=10.1 Hz, 1H), 6.15 (dd, J=10.1, 1.9 Hz, 1H), 6.00 (t, J=1.7 Hz, 1H), 4.86 (d, J=7.1 Hz, 1H), 4.68 (d, J=7.1 Hz, 1H), 4.19 (s, 1H), 3.37 (s, 3H), 2.90-2.67 (m, 4H), 2.41 (tdd, J=13.5, 5.1, 1.6 Hz, 1H), 2.29 (ddd, J=13.3, 4.4, 2.4 Hz, 1H), 2.08-1.69 (m, 7H), 1.68-1.54 (m, 4H), 1.50 (s, 3H), 1.28-1.06 (m, 5H), 1.02-0.90 (m, 3H), 0.87 (s, 3H); ¹³C NMR (126 MHz, CDCl₃): δ 186.32, 169.31, 155.88, 127.42, 123.76, 92.25, 81.59, 61.57, 56.65, 56.59, 55.25, 52.18, 43.55, 43.39, 39.81, 34.89, 33.50, 32.88, 32.29, 31.69, 26.40, 24.06, 22.70, 22.29, 21.96, 18.67, 13.47; HRMS (ESI-TOF): Calcd for C₂₇H₄₀NaO₃S₂ ([M+Na]⁺): 499.2311; Found: 499.2298.

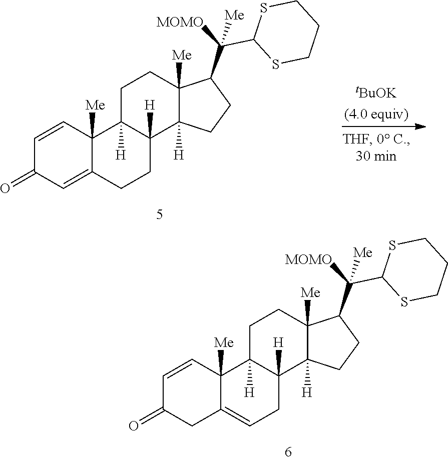

tBuOK (2.0 M in THF, 177.5 mL, 354.8 mmol, 4.0 equiv) was added dropwise to the solution of compound 5 (42.3 g, 88.7 mmol) in dry THF (400 mL) at 0° C. The reaction was stirred at room temperature for 30 min. Then the reaction mixture was poured into cold saturated NH₄Cl solution. The layers were separated, and the aqueous layer was extracted with Et₂O three times. The combined organic layers were washed with brine, dried with Na₂SO₄, and concentrated in vacuo. Flash column chromatography (silica gel, hexanes: EtOAc=5:1 to 3:1) afforded compound 6 (32 g, 76% yield).

Physical State: white foamy solid; R_{f}=0.55 (2:1 hexane: EA); [α]_{D}^{23.5}+75.6 (c 1.0, CHCl₃); ¹H NMR (500 MHz, Chloroform-d): δ 6.91 (dd, J=10.2, 3.9 Hz, 1H), 5.81 (dd, J=10.5, 4.8 Hz, 1H), 5.38 (s, 1H), 4.86 (t, J=5.6 Hz, 1H), 4.68 (t, J=5.9 Hz, 1H), 4.21 (d, J=4.3 Hz, 1H), 3.36 (d, J=4.9 Hz, 3H), 3.32-3.24 (m, 1H), 2.89-2.66 (m, 5H), 2.13-1.91 (m, 4H), 1.84 (t, J=7.1 Hz, 211), 1.74 (d, J=11.5 Hz, 1H), 1.69-1.62 (m, 1H), 1.62-1.45 (m, 7H), 1.26 (t, J=12.9 Hz, 1H), 1.15 (d, J=4.3 Hz, 3H), 1.13-1.09 (m, 2H), 1.01 (dq, J=12.0, 6.4, 5.9 Hz, 1H), 0.86 (d, J=4.7 Hz, 3H); ¹³C NMR (126 MHz, CDCl₃): δ 198.24, 156.08, 135.71, 126.40, 123.49, 92.23, 81.65, 61.58, 56.62, 56.58, 56.42, 45.35, 45.22, 43.05, 39.83, 39.77, 32.26, 31.67, 31.29, 30.90, 26.39, 23.80, 22.32, 21.96, 20.80, 19.16, 13.39; HRMS (ESI-TOF): Calcd for C₂₇H₄₀NaO₃S₂ ([M+Na]⁺): 499.2311; Found: 499.2302.

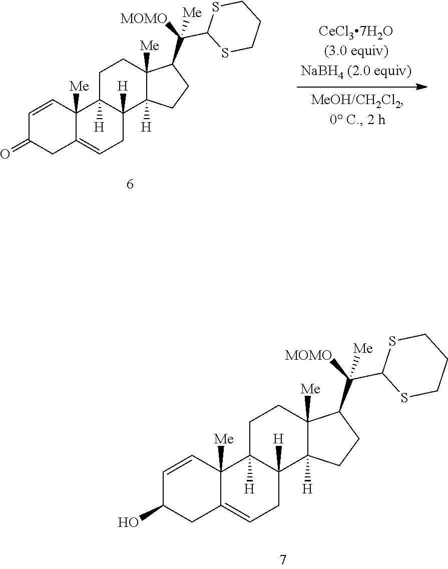

Added CeCl₃·7H₂O (75 g, 201.2 mmol, 3.0 equiv) and compound 6 (32 g, 67.1 mmol, 1.0 equiv) in a 1000 mL round bottom flask. Dissolved the mixture with MeOH (400 mL). The resulting mixture was stirred at room temperature for 30 min, then cooled the temperature to 0° C. with an ice bath. Added NaBH₄ (5.1 g, 134.2 mmol, 2.0 equiv) portion wise in 1 h. Kept stirring at 0° C. for 2 h. Monitored the reaction by TLC until completion. Added cold H₂O carefully into the system to quench the reaction under 0° C. Removed most of the MeOH under reduced pressure by rotary evaporation. The aqueous layer was extracted with EtOAc and monitored by TLC until no product showed on TLC. The combined organic layers were washed with brine, dried with Na₂SO₄, filtered, and concentrated in vacuo. Flash column chromatography (silica gel, hexanes: EtOAc=5:1 to 3:1 to 1:1) afforded compound 7 (28.7 g, 90% yield).

Physical State: white solid; R_{f}=0.31 (2:1 hexane: EA); [α]_{D}^{22.9}+11.6 (c 1.0, CHCl₃); ¹H NMR (500 MHz, Chloroform-d): δ 5.73 (dt, J=10.0, 1.9 Hz, 1H), 5.51 (dd, J=10.3, 1.8 Hz, 1H), 5.38 (dd, J=4.5, 2.2 Hz, 1H), 4.89 (dd, J=7.1, 1.3 Hz, 1H), 4.72 (dd, J=7.2, 1.4 Hz, 1H), 4.24 (d, J=1.4 Hz, 1H), 4.17 (ddd, J=10.0, 6.0, 1.9 Hz, 1H), 3.40 (d, J=1.4 Hz, 3H), 2.91-2.73 (m, 4H), 2.47-2.38 (m, 1H), 2.32-2.21 (m, 1H), 2.10-1.94 (m, 4H), 1.91-1.73 (m, 4H), 1.67-1.44 (m, 8H), 1.25 (td, J=12.8, 4.1 Hz, 1H), 1.20-1.07 (m, 1H), 1.06 (d, J=1.4 Hz, 3H), 1.04-0.96 (m, 2H), 0.86 (d, J=1.5 Hz, 3H); ¹³C NMR (126 MHz, CDCl₃): δ 138.94, 136.36, 129.46, 121.85, 92.32, 81.87, 69.78, 61.76, 56.75, 56.73, 56.66, 46.66, 43.19, 40.46, 40.07, 38.52, 32.41, 31.78, 31.29, 31.19, 26.51, 23.95, 22.47, 21.98, 21.89, 20.98, 13.53. HRMS (ESI-TOF): Calcd for C₂₇H₄₂NaO₃S₂ ([M+Na]⁺): 501.2468; Found: 501.2454.

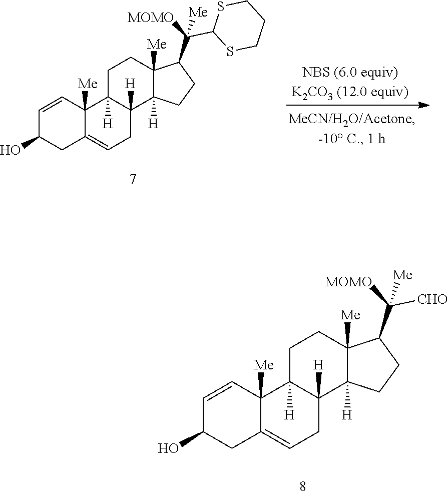

Freshly crystallized N-bromosuccinimide (8.9 g, 50.1 mmol, 6.0 equiv) was dissolved in MeCN (90 mL). A solution of K₂CO₃ (13.9 g, 100.3 mmol, 12.0 equiv) in water (25 mL) was added, and the reaction flask was cooled to −10° C. A solution of dithiane 7 (4.0 g, 8.4 mmol, 1.0 equiv) in acetone (40 mL) was added. The resulting mixture was stirred at −10° C. for 1 hour. Monitored the. Reaction by ¹H-NMR. Took aliquot from the system into a round bottom flask. Dried it by rotary evaporation and prepared the NMR sample directly. After completion, sat. aq. Na₂SO₃ was added until the yellow color disappeared. The aqueous layer was extracted with EtOAc and the combined organic layers were washed with brine (50 mL), dried over anhydrous Na₂SO₄, filtered, and concentrated under reduced pressure by rotary evaporation. Flash column chromatography (silica gel, hexanes: EtOAc=50:1 to 3:1 to 2:1) afforded aldehyde 8 (2.6 g, 80% yield).

Physical State: white solid; R_{f}=0.31 (2:1 hexane: EA); [α]_{D}^{22.6}+9.5 (c 1.0, CHCl₃); ¹H NMR (500 MHz, Chloroform-d): δ 9.66 (s, 1H), 5.72 (d, J=10.1 Hz, 1H), 5.51 (d, J=10.1 Hz, 1H), 5.43-5.28 (m, 1H), 4.81 (d, J=7.2 Hz, 1H), 4.59 (d, J=7.3 Hz, 1H), 4.16 (dd, J=9.6, 5.9 Hz, 1H), 3.38 (s, 3H), 2.42 (dd, J=12.1, 6.0 Hz, 1H), 2.25 (t, J=11.1 Hz, 1H), 2.10 (dd, J=20.2, 7.4 Hz, 2H), 1.97 (dd, J=12.6, 4.7 Hz, 1H), 1.84 (q, J=10.5 Hz, 1H), 1.72-1.41 (m, 7H), 1.33 (s, 3H), 1.19 (td, J=13.2, 4.5 Hz, 1H), 1.15-0.93 (m, 6H), 0.76 (s, 3H); ¹³C NMR (126 MHz, CDCl₃): δ 205.36, 139.06, 136.15, 129.62, 121.59, 91.95, 85.61, 69.70, 58.47, 56.19, 55.87, 46.75, 43.41, 40.40, 39.86, 38.51, 31.42, 31.18, 23.91, 21.92, 21.89, 20.89, 18.81, 14.79. HRMS (ESI-TOF): Calcd for C₂₄H₃₆NaO₄ ([M+Na]⁺): 411.2506; Found: 411.2496.

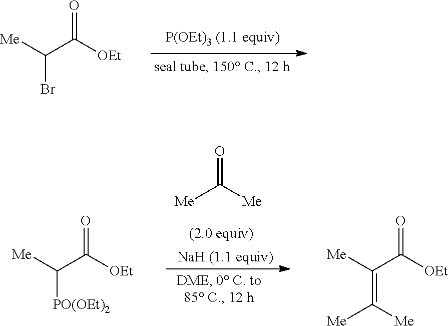

Ethyl 2-bromopropionate (15.8 mL, 121.8 mmol, 1.0 equiv was mixed with triethylphosphite (23 mL, 134 mmol, 1.1 equiv) and heated to 150° C. overnight in a seal tube. After cooling to room temperature, the mixture was transferred to a round bottom flask and azeotroped with toluene three times on a rotary evaporator to remove volatiles. After that, the clear liquid (30.9 g) was used directly in the next step.

Triethyl 2-phosphonopropionate (30.9 g, 122 mmol, 1.0 equiv) was added dropwise to a stirred solution of NaH (60% in mineral oil, 5.4 g, 134 mmol, 1.1 equiv) in dry dimethoxyethane (200 mL) at 0° C. During this process, a lot of gas would generate in the system. After stirring at room temperature for 30 min, acetone (17.9 mL, 244 mmol, 2.0 equiv) was added and the reaction mixture was stirred at 85° C. overnight. The reaction mixture was cooled, diluted with water and extracted with ether. The combined organic layers were dried over anhydrous Na₂SO₄, filtered and concentrated. The residue was purified by flash column chromatography on silica gel with hexane/ether (1:0 to 20:1, stained in KMnO₄) to give α,β-unsaturated carboxylic ester (9.2 g, 53% yield) as a colorless oil with fruit fragrance.

R_{f}=0.55 (5:1 hexane: ether); ¹H NMR (500 MHz, Chloroform-d): δ 4.18 (q, J=7.1 Hz, 2H), 2.00 (q, J=1.5 Hz, 3H), 1.85 (dt, J=2.8, 1.4 Hz, 3H), 1.80 (q, J=1.0 Hz, 3H), 1.30 (t, J=7.1 Hz, 3H).

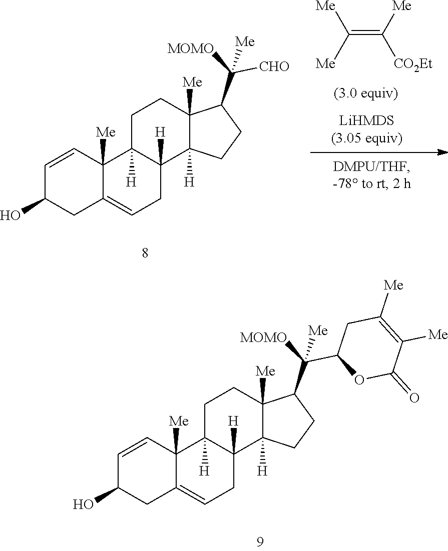

To a solution of HMDS (7.2 mL, 34.1 mmol, 3.3 equiv) in dry THF (26 mL) in a 500 mL round bottom flask was added ⁿBuLi (1.66 M in hexane, 19 mL, 31.5 mmol, 3.05 equiv) dropwise at 0° C. The mixture was stirred at 0° C. for 30 min. Then cooled the flask to −78° C. In a 100 mL round bottom flask was added 2,3-dimethylbut-2-enoate (4.4 g, 31 mmol, 3.0 equiv), DMPU (26 mL) and THF (26 mL). The mixture was added dropwise to the solution of LiHMDS at −78° C. (dry ice-acetone bath) and the mixture was stirred for 1.5 h at that temperature. A solution of the aldehyde 8 (4.02 g, 10.34 mmol, 1.0 equiv) in THF was added dropwise and the resulting mixture was stirred at −78° C. for 1 h. After that, the temperature was allowed to increase to room temperature in 1 h by removing the dry ice-acetone bath. During the process, the reaction mixture became pink and was monitored by TLC until completion. The reaction was quenched by addition of saturated NH₄Cl solution. The mixture was extracted with Et₂O. the combined organic layers were washed with brine, dried over Na₂SO₄, filtered, and concentrated under reduced pressure on a rotary evaporator. The residue was purified by flash column chromatography (hexane:EtOAc=5:1 to 3:1) to afford lactone 9 (4.1 g, 78% yield) as a white foamy solid.

Physical State: white foamy solid; R_{f}=0.19 (2:1 hexane: EA); [α]_{D}^{23.2}+57.0 (c 1.0, CHCl₃); ¹H NMR (500 MHz, CDCl₃) δ 5.77 (dd, J=10.1, 2.0 Hz, 1H), 5.54 (dt, J=10.1, 1.7 Hz, 1H), 4.99 (d, J=6.6 Hz, 1H), 4.83 (d, J=6.6 Hz, 1H), 4.25 (dd, J=13.3, 3.4 Hz, 1H), 4.21 (ddt, J=9.7, 5.9, 1.8 Hz, 1H), 3.38 (s, 3H), 2.53-2.42 (m, 2H), 2.29 (tq, J=12.0, 2.3 Hz, 1H), 2.18-2.10 (m, 1H), 2.07-1.98 (m, 3H), 1.94 (t, J=1.1 Hz, 3H), 1.87 (dt, J=2.3, 1.1 Hz, 3H), 1.69-1.47 (m, 7H), 1.41 (s, 3H), 1.35-1.13 (m, 2H), 1.09 (s, 3H), 1.07-0.99 (m, 2H), 0.89 (s, 3H). ¹³C NMR (126 MHz, CDCl₃) δ 166.22, 148.99, 139.12, 136.14, 129.60, 121.87, 121.55, 92.76, 82.26, 79.89, 69.63, 56.77, 56.29, 54.54, 46.65, 43.06, 40.35, 40.13, 38.49, 32.15, 31.20, 23.91, 21.88, 20.89, 20.54, 17.80, 13.86, 12.47. HRMS (ESI-TOF): Calcd for C₃₀H₄₄NaO₅ ([M+Na]⁺): 507.3081; Found: 507.3068.

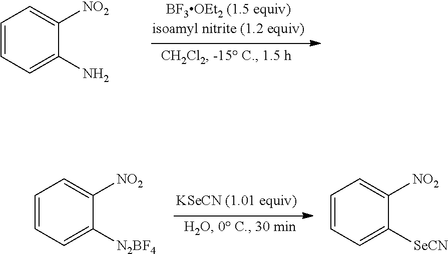

To a solution of BF₃·Et₂O (20.6 mL, 163 mmol, 1.5 equiv), cooled at −15° C., was added a solution of o-nitroaniline (15 g, 108.6 mmol, 1.0 equiv) in 150 mL of dry CH₂Cl₂. The suspension was stirred at −15° C. for 15 min. Then a solution of isoamyl nitrite (17.5 mL, 130 mmol, 1.2 equiv) in 75 nL of dry CH₂Cl₂ was added dropwise to the vigorously stirred reaction mixture. After complete addition of the reactant, this mixture was stirred at −15° C. for 30 min and then at 0° C. for 30 min. Cold pentane (120 mL) was added to the suspension and the precipitate was filtered off and washed with cold dry Et₂O (300 mL). Diazonium salt was obtained in quantitative yield as a pale-yellow powder and used without further purification.

¹H NMR (500 MHz, DMSO) δ 9.14 (dd, J=8.1, 1.4 Hz, 1H), 8.83 (dd, J=8.3, 1.2 Hz, 1H), 8.57 (td, J=8.0, 1.4 Hz, 1H), 8.45 (td, J=7.9, 1.2 Hz, 1H).

To a solution of 2-nitrobenzenediazonium tetrafluoroborate (13 g, 55 mmol, 1.0 equiv) in cold water (300 mL) was added a solution of KSeCN (8.0 g, 55.5 mmol, 1.01 equiv) in 80 mL of water dropwise slowly at 0° C. After complete addition of KSeCN, the suspension was stirred at 0° C. for 10 min and the precipitate was filtered off and washed with H₂O. The crude product (11.8 g) was dried on lyophilizer overnight and used in the next step without further purification.

¹H NMR (500 MHz, CDCl₃) δ 8.45 (dd, J=8.3, 1.5 Hz, 1H), 8.22 (dd, J=1 t Hz, 1H), 7.78 (ddd, J=8.3, 7.3, 1.5 Hz, 1H), 7.61 (ddd, J=8.4, 7.2, 1.2 Hz, 1H).

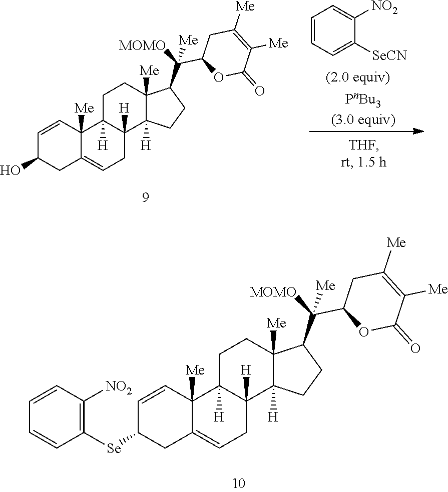

To a solution of alcohol 9 (5.1 g, 10.5 mmol, 1.0 equiv) and o-nitrophenyl selenocyanate (4.64 g, 21 mmol, 2.0 equiv) in anhydrous THE (125 mL) was added tri-n-butylphosphine (7.9 mL, 31.5 mmol, 3.0 equiv) dropwise. The resulting mixture turned dark purple and was stirred at rt for 1.5 h and then absorbed onto silica gel to prepare dry sample to load on to column for purification (hexane:EtOAc=4:1) to give selenide 10 (6.9 g, 98% yield).

Physical State: yellow foamy solid; R_{f}=0.31 (2:1 hexane: EA); [α]_{D}^{23.3}−26.05 (c 1.0, CHCl₃); ¹H NMR (500 MHz, Chloroform-d): δ 8.16 (dd, J=8.2, 1.7 Hz, 1H), 7.56 (d, J=8.1 Hz, 1H), 7.50-7.43 (m, 1H), 7.30-7.15 (m, 1H), 5.83 (dd, J=9.8, 3.2 Hz, 1H), 5.73 (dd, J=9.8, 5.3 Hz, 1H), 5.30 (d, J=4.7 Hz, 1H), 4.96 (dd, J=6.7, 2.2 Hz, 1H), 4.76 (dd, J=6.7, 2.2 Hz, 1H), 4.19 (dd, J=13.4, 3.3 Hz, 1H), 4.11 (t, J=5.2 Hz, 1H), 3.31 (s, 3H), 3.06-2.95 (m, 1H), 2.32 (p, J=14.5, 13.3 Hz, 2H), 2.10 (dd, J=17.5, 3.4 Hz, 1H), 2.04-1.90 (m, 4H), 1.88 (s, 3H), 1.79 (s, 3H), 1.59-1.52 (m, 5H), 1.48-1.44 (m, 3H), 1.35 (s, 3H), 1.20 (td, J=12.9, 4.5 Hz, 1H), 1.10 (dq, J=14.2, 8.8, 6.8 Hz, 1H), 1.00 (m, 5H), 0.84 (d, J=2.8 Hz, 3H). ¹³C NMR (126 MHz, CDCl₃): δ 166.19, 148.97, 146.93, 139.05, 136.94, 134.48, 133.67, 129.57, 126.45, 125.45, 123.47, 123.31, 121.78, 92.97, 82.37, 79.81, 56.65, 56.34, 55.00, 46.42, 43.13, 40.58, 40.20, 38.80, 35.77, 32.27, 31.17, 30.98, 23.90, 21.79, 21.65, 20.88, 20.53, 17.33, 13.71, 12.45. HRMS (ESI-TOF): Calcd for C₃₆H₄₇NNaO₆Se ([M+Na]⁺): 692.2461; Found: 692.2460.

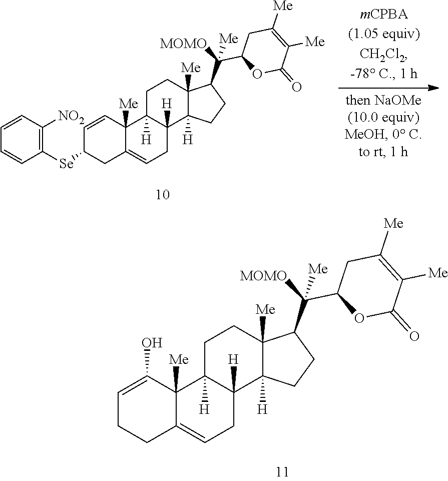

To a solution of selenide 10 (8.2 g, 12.23 mmol, 1.0 equiv) in non-anhydrous CH₂Cl₂ (50 mL) at −78° C. was added dropwise a solution of mCPBA (2.96 g, 12.84 mmol, 1.05 equiv) in 30 mL CH₂Cl₂ over 30 min. Stirred at −78° C. for 1 h. Took aliquot from the system and dried on rotary evaporator. Prepared the NMR sample and monitored the reaction with ¹H-NMR. Added MeOH (150 mL) into the reaction system at 0° C. Then added NaOMe (6.61 g, 122.3 mmol, 10.0 equiv) in one portion at that temperature. The reaction mixture turned dark immediately. Stirred at 0° C. for 1 h. Monitored the reaction with TLC. Upon completion, the reaction mixture was dried on rotary evaporator, the residue partitioned between Et₂O and H₂O. Exacted with Et₂O. Combined the organic phase and dried with Na₂SO₄, filtered, and concentrated in vacuo. The residue was purified by flash column chromatography (hexane:EtOAc=5:1 to 3:1) to afford compound 11 (4.4 g, 74% yield) as a yellow foamy solid.

Physical State: yellow foamy solid; R_{f}=0.16 (2:1 hexane: EA); [α]_{D}^{22.4}+72.1 (c 1.0, CHCl₃); ¹H NMR (500 MHz, Chloroform-d): δ 5.91 (ddd, J=9.9, 5.3, 2.6 Hz, 1H), 5.75 (dt, J=9.8, 3.5 Hz, 1H), 5.58 (d, J=5.5 Hz, 1H), 4.98 (d, J=6.6 Hz, 1H), 4.81 (d, J=6.6 Hz, 1H), 4.24 (dd, J=13.4, 3.4 Hz, 1H), 3.75 (d, J=5.6 Hz, 1H), 3.35 (s, 3H), 2.98-2.84 (m, 1H), 2.75-2.59 (m, 1H), 2.48-2.33 (m, 1H), 2.10 (dd, J=17.5, 3.4 Hz, 1H), 2.05-1.94 (m, 3H), 1.91 (s, 3H), 1.84 (s, 3H), 1.82-1.80 (m, 1H), 1.67-1.54 (m, 3H), 1.53-1.43 (m, 4H), 1.39 (s, 3H), 1.33 (td, J=12.3, 4.8 Hz, 1H), 1.23-1.00 (m, 3H), 0.91 (s, 3H), 0.87 (s, 3H). ¹³C NMR (126 MHz, CDCl₃): δ 166.18, 148.76, 135.60, 129.12, 127.75, 124.14, 121.94, 92.92, 82.32, 79.92, 69.83, 56.60, 56.34, 54.92, 43.04, 42.21, 40.71, 40.08, 32.24, 32.03, 31.93, 31.75, 24.07, 21.80, 20.55, 20.31, 18.80, 17.54, 13.72, 12.49. HRMS (ESI-TOF): Calcd for C₃₀H₄₄NaO₅ ([M+Na]⁺): 507.3081; Found: 507.3076.

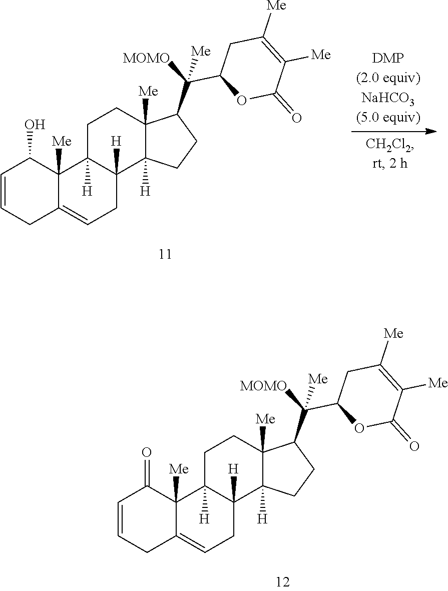

To a stirred solution of compound 11 (5.3 g, 10.93 mmol, 1.0 equiv) and NaHCO₃ fine powder (4.6 g, 54.2 mmol, 5.0 equiv) in CH₂Cl₂ (100 mL) at room temperature was added Dess-Martin periodinane (9.2 g, 21.7 mmol, 2.0 equiv). The resulting mixture was stirred at room temperature for 2 h. Monitored the reaction with TLC. Upon completion, aq. saturated NaHCO₃ and Na₂S₂O₃ were added sequentially. The mixture was stirred for 20 min. Phases were separated then, Extracted the aqueous phase with CH₂Cl₂. The organic phase was combined and washed with brine. The organic phase was dried over Na₂SO₄, filtered, and concentrated under vacuum. Flash column chromatography (silica gel, hexanes: EtOAc=5:1) afforded compound 12 (4.5 g, 86% yield) as a pale yellow solid.

Physical State: pale yellow solid; R_{f}=0.38 (2:1 hexane: EA); [α]_{D}^{22.9}+35.4 (c 1.0, CHCl₃); ¹H NMR (500 MHz, Chloroform-d): δ 6.73 (ddd, J=10.1, 5.1, 2.5 Hz, 1H), 5.82 (dd, J=9.9, 3.0 Hz, 1H), 5.53 (d, J=5.9 Hz, 1H), 4.97 (d, J=6.6 Hz, 1H), 4.80 (d, J=6.6 Hz, 1H), 4.22 (dd, J=13.3, 3.5 Hz, 1H), 3.34 (s, 3H), 3.30-3.19 (m, 1H), 2.79 (dd, J=21.3, 5.0 Hz, 1H), 2.40 (t, J=15.6 Hz, 1H), 2.13 (ddd, J=30.4, 15.5, 3.7 Hz, 2H), 2.04-1.92 (m, 3H), 1.91 (s, 3H), 1.83 (s, 3H), 1.65-1.40 (m, 8H), 1.39 (s, 3H), 1.19 (s, 3H), 1.14-0.98 (m, 2H), 0.87 (s, 3H). ¹³C NMR (126 MHz, CDCl₃): δ 204.38, 166.12, 148.71, 145.21, 135.97, 127.97, 124.75, 121.90, 92.94, 82.32, 79.84, 56.54, 56.30, 55.10, 50.57, 42.98, 42.90, 40.38, 33.52, 32.61, 32.24, 30.74, 23.99, 23.46, 21.60, 20.51, 18.99, 17.41, 13.81, 12.46. HRMS (ESI-TOF): Calcd for C₃₀H₄₂NaO₆ ([M+Na]⁺): 505.2924; Found: 505.2920.

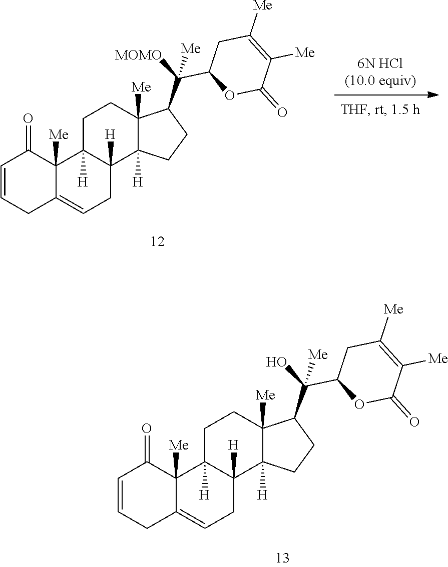

To a solution of compound 12 (3.35 g, 6.94 mmol, 1.0 equiv) in THF at room temperature was added 6 N HCl (11.5 mL, 69.4 mmol, 10.0 equiv) dropwise. After that, the mixture was stirred at room temperature for 1.5 h. Monitored the reaction with TLC. Upon completion, the reaction was quenched by adding aq. saturated NaHCO₃. Aqueous phase was extracted with CH₂Cl₂. Organic phase was combined, dried with Na₂SO₄, filtered, and concentrated on rotary evaporator. The residue was purified by flash column chromatography (hexane:EtOAc=4:1 to 3:1) to afford compound 13 (2.0 g, 64% yield) as white foamy solid.

Physical State: white foamy solid; R_{f}=0.20 (2:1 hexane: EA); [α]_{D}^{25.0}+20.9 (c 1.0, CHCl₃); ¹H NMR (500 MHz, Chloroform-d): δ 6.73 (ddd, J=10.0, 5.0, 2.5 Hz, 1H), 5.82 (dd, J=10.0, 2.9 Hz, 1H), 5.57-5.46 (m, 1H), 4.18 (dd, J=13.3, 3.5 Hz, 1H), 3.25 (dq, J=21.4, 2.9 Hz, 1H), 2.79 (dd, J=21.3, 5.0 Hz, 1H), 2.48-2.32 (m, 1H), 2.20-1.97 (m, 4H), 1.92 (s, 5H), 1.85 (s, 3H), 1.59 (td, J=11.0, 3.7 Hz, 2H), 1.54-1.30 (m, 6H), 1.27 (s, 3H), 1.19 (s, 3H), 1.15-0.98 (m, 2H), 0.87 (s, 3H). ¹³C NMR (126 MHz, CDCl₃): δ 204.46, 166.13, 149.01, 145.27, 135.94, 127.94, 124.74, 121.93, 81.06, 75.23, 56.69, 54.71, 50.57, 42.98, 42.93, 40.21, 33.53, 32.62, 31.63, 30.72, 23.92, 23.49, 21.81, 21.03, 20.58, 19.02, 13.71, 12.50. HRMS (ESI-TOF): Calcd for C₂₈H₃₉O₄ ([M+H]³⁰): 439.2843; Found: 439.2846.

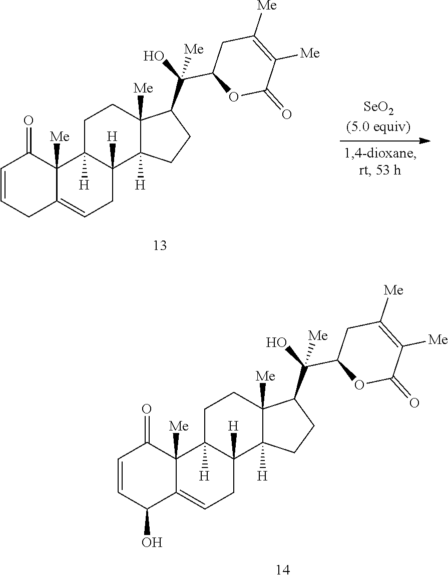

SeO₂ (1.4 g, 12.5 mmol, 5.0 equiv) was added to a solution of compound 13 (1.1 g, 2.5 mmol, 1.0 equiv) in 1,4-dioxane (50 mL) at room temperature. The mixture was stirred at room temperature for 53 h. During this process, monitored the reaction by LC-MS. When the LC-MS yield based on the integration reached about 76%, stopped the reaction. There was a bit starting material left. Added silica gel to the reaction mixture and concentrated on rotary evaporator to prepare dry sample to load it onto column directly. Flash column chromatography (hexane:EtOAc=2:1 to 1:1) afforded compound 14 (807 mg, 71% yield) as a single diastereomer and recovered starting material (200 mg, 82% conversion).

Physical State: pale yellow powder; R_{f}=0.29 (1:1 hexane: EA twice); [α]_{D}^{22.7}+62.7 (c 1.0, CHCl₃); ¹H NMR (500 MHz, Chloroform-d): δ 6.76 (dd, J=10.0, 4.5 Hz, 1H), 5.98-5.90 (m, 2H), 4.62 (d, J=4.5 Hz, 1H), 4.22 (dd, J=13.4, 3.5 Hz, 1H), 2.48-2.36 (m, 1H), 2.18-2.03 (m, 4H), 1.95 (s, 4H), 1.92-1.84 (m, 3H), 1.69-1.47 (m, 8H), 1.45 (s, 3H), 1.43-1.35 (m, 1H), 1.30 (s, 3H), 1.21 (ddd, J=24.6, 11.3, 4.3 Hz, 1H), 1.11 (ddd, J=12.9, 9.8, 6.8 Hz, 1H), 0.91 (s, 3H). ¹³C NMR (126 MHz, CDCl₃): δ 203.80, 166.26, 149.15, 143.24, 138.85, 130.98, 129.00, 121.97, 81.09, 75.26, 69.34, 56.69, 54.73, 49.42, 43.01, 42.85, 40.10, 32.12, 31.64, 31.04, 23.98, 23.01, 22.91, 21.88, 21.01, 20.64, 13.77, 12.52. HRMS (ESI-TOF): Calcd for C₂₈H₃₈NaO₅ ([M+Na]⁺): 477.2611; Found: 427.2615

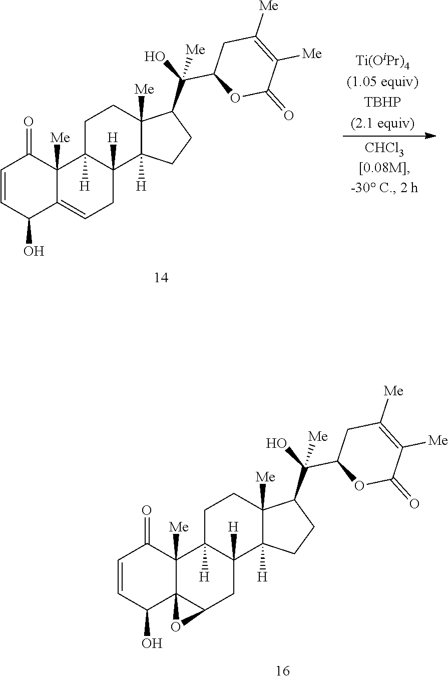

Compound 14 (1.18 g, 2.6 mmol, 1.0 equiv) was dissolved in dry CHCl₃ (32 mL). The mixture was cooled to −30° C. Then titanium (IV) isopropoxide (0.82 mL, 2.72 mmol, 1.05 equiv) was added dropwise. The resulting solution was stirred at −30° C. for 5 min and added tert-butyl hydroperoxide (5.5 M in decane, 1.0 mL, 5.44 mmol, 2.1 equiv). After 2 h of stirring, H₂O was added and kept stirring until the precipitation was complete. The resulting mixture was loaded onto column directly. Flash column chromatography (silica gel, hexanes: EtOAc=1:1) afforded compound 16 (920 mg, 75.4% yield).

Physical State: white solid; R_{f}=0.15 (1:1 hexane: EA, twice); [α]_{D}^{23.41}+89.5 (c 1.0, CHCl₃); ¹H NMR (500 MHz, Chloroform-d): δ 6.92 (dd, J=10.0, 5.9 Hz, 1H), 6.18 (d, J=10.0 Hz, 1H), 4.17 (dd, J=13.3, 3.5 Hz, 1H), 3.73 (d, J=5.9 Hz, 1H), 3.20 (s, 1H), 2.99-2.82 (m, 1H), 2.41 (t, J=15.6 Hz, 1H), 2.33 (s, 1H), 2.16-2.10 (m, 1H), 2.07 (dd, J=17.6, 3.5 Hz, 1H), 1.98-1.89 (m, 5H), 1.85 (s, 3H), 1.76 (dt, J=11.7, 3.6 Hz, 1H), 1.64-1.58 (m, 1H), 1.55-1.42 (m, 4H), 1.38 (s, 3H), 1.27-1.22 (m, 4H), 1.20-1.11 (m, 2H), 0.97-0.89 (m, 2H), 0.82 (s, 3H). ¹³C NMR (126 MHz, CDCl₃): δ 202.28, 166.17, 149.08, 142.10, 132.49, 122.01, 80.94, 75.16, 70.02, 64.01, 62.31, 56.61, 54.69, 47.81, 44.14, 42.81, 39.73, 31.54, 31.17, 29.27, 23.87, 21.99, 21.87, 20.81, 20.65, 17.31, 13.57, 12.53. HRMS (ESI-TOF): Calcd for C₂₈H₃₈NaO₆ ([M+Na]⁺): 493.2561; Found: 493.2560.

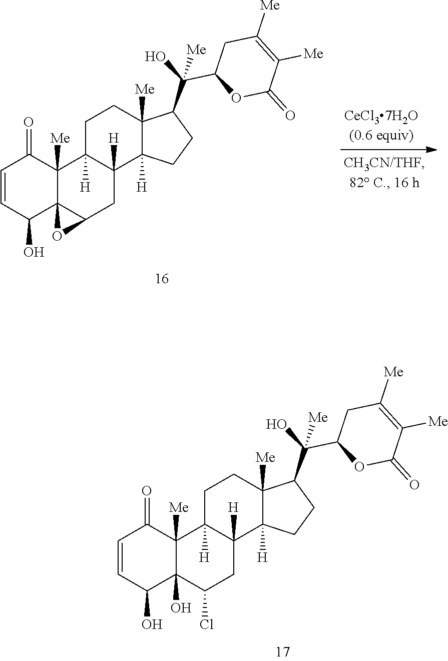

A solution of CeCl₃·7H₂O (4.7 mg, 0.013 mmol, 0.6 equiv) in dry CH₃CN (0.5 mL) was sonicated for 1 h and then added dropwise to a solution of compound 16 (10 mg, 0.021 mmol, 1.0 equiv) in a mixture of CH₃CN/THF (1:1, 2 mL), at room temperature under argon. The reaction mixture was heated to 82° C. for 12 h, and afterwards, silica gel was added to the system and prepared the dry sample to load it on to column directly. Flash column chromatography (silica gel, hexanes: EtOAc=1:1) afforded compound 17 (9.1 mg, 85.5% yield).

Physical State: white solid; R_{f}=0.375 (1:2 hexane: EA); [α]_{D}^{24.56}+56.6 (c 0.89, CHCl₃); ¹H NMR (500 MHz, Chloroform-d): δ 6.47 (dd, J=10.4, 2.6 Hz, 1H), 6.00 (dd, J=10.4, 2.2 Hz, 111), 5.03 (dt, J=4.7, 2.5 Hz, 111), 4.44 (dd, J=12.4, 4.8 Hz, 111), 4.19 (dd, J=13.2, 3.5 Hz, 1H), 3.73 (s, 1H), 3.13 (d, J=4.5 Hz, 111), 2.46 (dddd, J=17.6, 13.2, 3.5, 1.6 Hz, 111), 2.37-2.30 (m, 1H), 2.19 (s, 1H), 2.08 (dd, J=17.5, 3.5 Hz, 1H), 1.99-1.90 (m, 4H), 1.91-1.88 (m, 1H), 1.87 (s, 3H), 1.73-1.54 (m, 5H), 1.40-1.27 (m, 3H), 1.25 (s, 3H), 1.22 (s, 3H), 1.20-1.07 (m, 2H), 0.96-0.86 (m, 1H), 0.82 (s, 3H). ¹³C NMR (126 MHz, CDCl₃): δ 200.27, 166.08, 149.10, 142.86, 127.93, 122.11, 80.81, 78.28, 75.09, 66.94, 66.33, 57.36, 55.90, 54.61, 45.91, 43.27, 39.44, 39.32, 34.62, 31.46, 23.62, 22.69, 22.10, 20.72, 20.63, 13.87, 12.59, 10.07. HRMS (ESI-TOF): Calcd for C₂₈H₄₀ClO₆ ([M+H]⁺): 507.2508; Found: 507.2496.

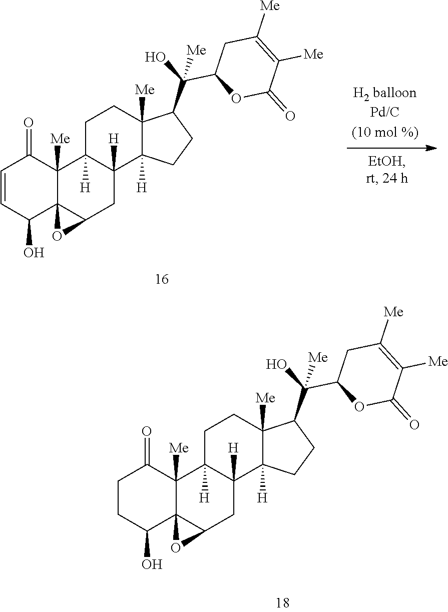

To a solution of compound 16 (10 mg, 0.021 mmol, 1.0 equiv) in EtOH was added 10% Pd/C (2.0 mg, 0.001 mmol, 10 mol %). The mixture was stirred under an atmosphere of H₂ for 24 h. After completion, added silica gel into the system and dried on the rotary evaporator and then loaded it onto the column directly. Flash column chromatography (silica gel, hexanes: EtOAc=1:1) afforded compound 18 (9.2 mg, 93% yield).

Physical State: white solid; R_{f}=0.14 (1:1 hexane: EA, twice); [α]_{D}^{24.57}−22.9 (c 1.25, CHCl₃); ¹H NMR (500 MHz, Chloroform-d): δ 4.19 (dd, J=13.3, 3.5 Hz, 1H), 3.51 (t, J=3.6 Hz, 1H), 3.13 (d, J=2.1 Hz, 1H), 2.62 (ddd, J=16.1, 9.1, 7.2 Hz, 1H), 2.52 (dt, J=10.7, 5.2 Hz, 1H), 2.48-2.40 (m, 1H), 2.36-2.31 (m, 1H), 2.26-2.17 (m, 2H), 2.11-2.07 (m, 2H), 2.04-1.99 (m, 1H), 1.94 (s, 3H), 1.87 (s, 3H), 1.70-1.60 (m, 2H), 1.59-1.50 (m, 2H), 1.47-1.37 (m, 1H), 1.37-1.31 (m, 2H), 1.30 (s, 3H), 1.24 (s, 3H), 1.22-1.14 (m, 2H), 1.12-1.07 (m, 1H), 1.01-0.84 (m, 2H), 0.80 (s, 3H). ¹³C NMR (126 MHz, CDCl₃): δ 211.58, 166.17, 149.08, 122.09, 80.94, 75.18, 72.97, 66.68, 59.02, 56.83, 54.73, 50.61, 43.12, 42.91, 39.49, 31.84, 31.54, 31.45, 28.95, 26.47, 23.88, 22.09, 21.40, 20.78, 20.70, 15.56, 13.57, 12.58. HRMS (ESI-TOF): Calcd for C₂₈H₄₁O₆ ([2M+H]⁺): 945.5723; Found: 945.5687.

TABLE 1
Screening of conditions for the conversion of 13 to 19.

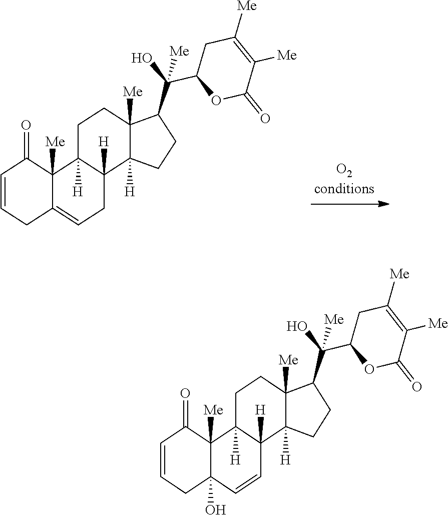

| entry | Photocatalyst (5 mol %) | solvent | Reducing agent | time | result |
|---|---|---|---|---|---|
| 1 | Rose bengal | CHCl₃ | — | 2 d | Super slow. |
| | | | | | Little conv. |
| 2 | Rose bengal | MeOH | — | 2 d | Super slow. |
| | | | | | Little conv. |
| 3 | Rose bengal | ⁱPrOH | — | 2 d | Super slow. |
| | | | | | Little conv. |
| 4 | Rose bengal | MeCN | PPh₃ | 24 h | Not full |
| | | | | | conv.; 22% |
| | | | | | yield |
| | | | | | determined |
| | | | | | by NMR; |
| | | | | | POPh₃ is |
| | | | | | mixed with |
| | | | | | product. |
| 5 | Rose bengal | MeCN | P(OEt)₃ | 12 h | Not full |
| | | | | | conv.; 25% |
| | | | | | yield |
| 6 | Rose bengal | acetone | P(OEt)₃ | 12 h | 29% yield |
| 7 | Rose bengal | pyridine | P(OEt)₃ | 24 h | 40% yield |
| 8 | Methylene | MeCN | P(OEt)₃ | 24 h | Not full |
| | blue | | | | conv.; 14% |
| | | | | | yield |
| 9 | TPP | pyridine | P(OEt)₃ | 12 h | 30% yield |
| 10 | Rose bengal | pyridine | P(OEt)₃ | 12 h, blue | Full conv. All |
| | | | | LED | decomposed. |
| 11 | Rose bengal | pyridine | PPh₃ | 3 d | Full conv. |
| | | | polymer | | 15% yield |
| 12 | Rose Bengal | pyridine | PPh₂Py | 45 h | 15.2% yield |
| | 50 mol % | | | | |
| | KBr as | | | | |
| | additive | | | | |
| 13 | Rose Bengal | pyridine | PPh₂Py | 45 h | 17.3% yield |

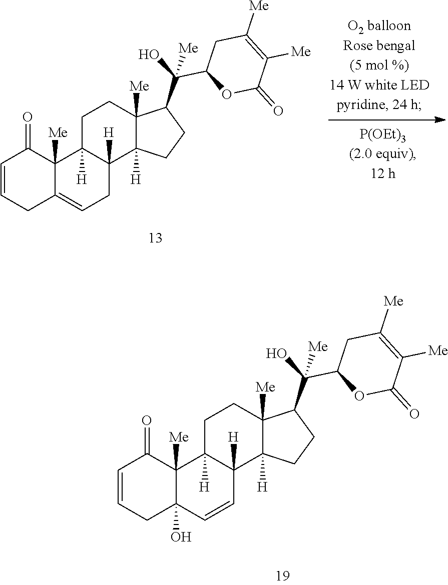

To a stirred solution of compound 13 (10 mg, 0.023 mmol, 1.0 equiv) in pyridine (1 mL) was added rose bengal (1.3 mg, 0.00115 mmol, 5 mol %) in a tube. The tube was charged with an oxygen balloon and irradiated with stirring using a 14 W white LED lamp at room temperature for 1 d. After that, P(OEt)₃ (7.9 μL, 0.046 mmol, 2.0 equiv) was added and the O₂ balloon was taken off. The mixture was stirred at room temperature for 12 h. The reaction was then concentrated in vacuo and purified by flash chromotography (silica gel, hexanes: EtOAc=1:1) to provide 19 (4.1 mg, 40% yield) as a white solid.

Physical State: white solid; R_{f}=0.16 (1:1 hexane: EA, twice); [α]_{D}^{24.34}+84.8 (c 0.88, CHCl₃); ¹H NMR (500 MHz, Chloroform-d): δ 6.58 (ddd, J=10.2, 5.2, 2.2 Hz, 1H), 5.89 (dd, J=10.2, 2.9 Hz, 1H), 5.73 (dd, J=9.8, 2.0 Hz, 1H), 5.57 (dd, J=9.8, 2.7 Hz, 1H), 4.21 (dd, J=13.4, 3.5 Hz, 1H), 2.76 (dd, J=8.9, 3.7 Hz, 1H), 2.62 (dt, J=19.1, 2.7 Hz, 1H), 2.47-2.29 (m, 3H), 2.10 (dt, J=13.7, 3.9 Hz, 2H), 1.95 (s, 3H), 1.89 (s, 3H), 1.85-1.82 (m, 1H), 1.77-1.70 (m, 2H), 1.65-1.35 (m, 7H), 1.31 (s, 4H), 1.19 (s, 3H), 0.94 (s, 3H). ¹³C NMR (126 MHz, CDCl₃): δ 204.11, 166.20, 148.99, 140.60, 133.37, 129.63, 129.42, 122.16, 81.12, 75.26, 75.05, 54.73, 54.31, 51.49, 44.21, 41.55, 40.71, 37.55, 36.61, 31.77, 23.71, 22.44, 21.95, 21.14, 20.71, 14.71, 13.94, 12.61. HRMS (ESI-TOF): Calcd for C₂₈H₃₈NaO₅ ([M+Na]⁺): 477.2611; Found: 477.2585.

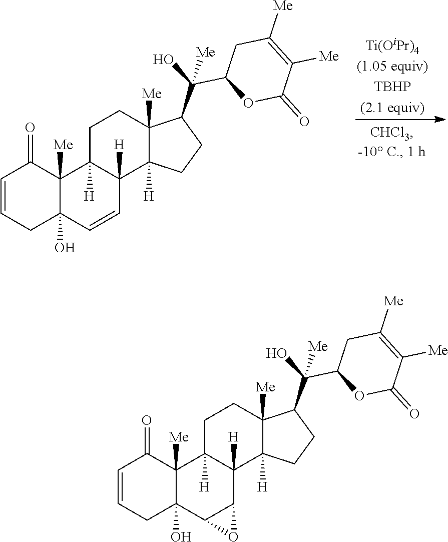

Compound (14.5 mg, 0.032 mmol, 1.0 equiv was dissolved in dry CHCl₃ (0.5 mL). The mixture was cooled to −10° C. Then titanium (IV) isopropoxide (10 μL, 0.033 mmol, 1.05 equiv) was added dropwise. The resulting solution was stirred at −10° C. for 5 min and added tert-butyl hydroperoxide (5.5 M in decane, 12.2 μL, 0.067 mmol, 2.1 equiv). After 1 h of stirring, H₂O was added and kept stirring until the precipitation was complete. The resulting mixture was loaded onto column directly. Flash column chromatography (silica gel, hexanes: EtOAc=1:1) afforded compound 20 (10.4 mg, 70% yield).

Physical State: white solid; R_{f}=0.10 (1:1 hexane: EA); [α]_{D}^{22.28}+75.4 (c 0.3, CHCl₃); ¹H NMR (500 MHz, Chloroform-d): δ 6.59 (ddd, J=10.2, 5.2, 2.2 Hz, 1H), 5.85 (dd, J=10.3, 2.8 Hz, 1H), 4.20 (dd, J=13.4, 3.5 Hz, 1H), 3.32 (dd, J=3.9, 2.1 Hz, 1H), 3.13 (d, J=1.5 Hz, 1H), 3.04 (d, J=3.8 Hz, 1H), 2.77-2.64 (m, 2H), 2.52 (dd, J=18.8, 5.2 Hz, 1H), 2.44-2.33 (m, 2H), 2.16-1.98 (m, 3H), 1.95 (s, 3H), 1.89 (s, 3H), 1.84 (tdd, J=9.4, 7.0, 3.7 Hz, 1H), 1.77 (td, J=10.5, 2.1 Hz, 1H), 1.59-1.33 (m, 7H), 1.32 (s, 3H), 1.18 (s, 3H), 0.96 (s, 3H). 13C NMR (126 MHz, CDCl₃): δ 203.33, 166.18, 148.97, 139.76, 129.15, 122.17, 81.15, 75.23, 73.40, 57.46, 56.46, 54.58, 52.06, 51.14, 44.00, 40.52, 36.88, 35.67, 35.18, 31.82, 23.34, 21.95, 21.87, 21.22, 20.71, 14.87, 13.90, 12.61. HRMS (ESI-TOF): Calcd for C₂₈H₃₉O₆ ([M+H]⁺): 471.2741; Found: 471.2710.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

1. A process for synthesizing Withanolide D having the structure 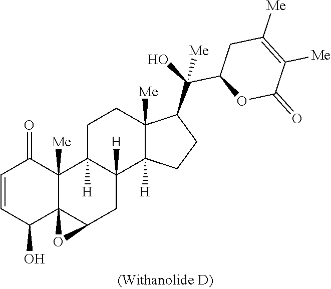
comprising treating a compound of Formula I 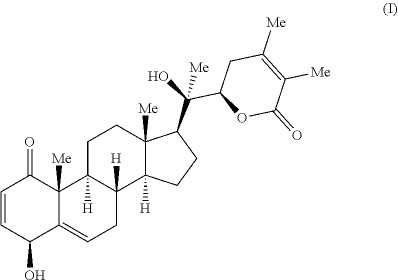
with a titanium alkoxide and tert-butyl hydroperoxide to provide Withanolide D.

2. The process of claim 1, further comprising preparing the compound of Formula I by a process comprising treating a compound of Formula II 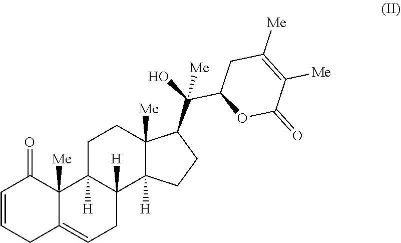
with selenium dioxide to provide the compound of Formula I.

3. A process for synthesizing Withanolide A having the structure 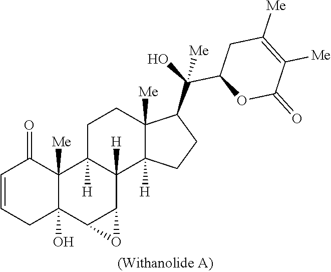
comprising treating Withacoagin having the structure 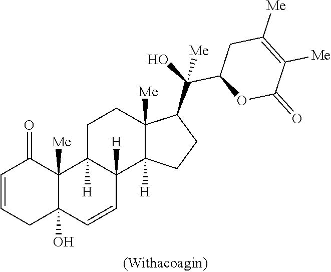
with a titanium alkoxide and tert-butyl hydroperoxide to provide Withanolide A.

4. The process of claim 3, further comprising preparing Withacoagin by a process comprising treating a compound of Formula II 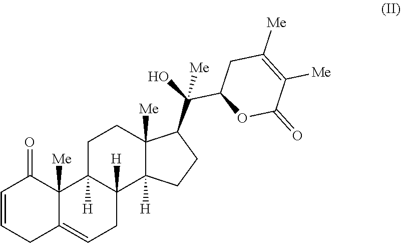
with light irradiation in the presence of a photocatalyst and oxygen followed by treatment with a reductant to provide Withacoagin.

5. The process of claim 2, further comprising preparing the compound of Formula II by a process comprising removing a protecting group PG from a compound of Formula III 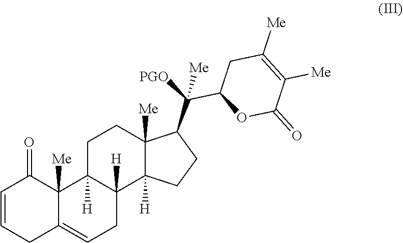
to provide a compound of Formula II.

6. The process of claim 5, further comprising preparing the compound of Formula III by a process comprising treating a compound of Formula IV 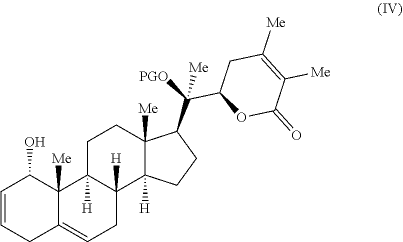
with an oxidant to provide the compound of Formula III.

7. The process of claim 6, further comprising preparing the compound of Formula IV by a process comprising treating a compound of Formula V 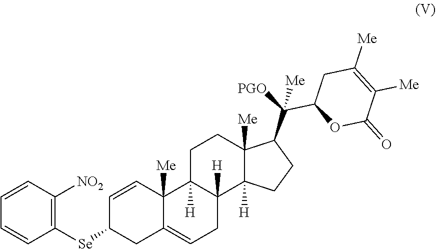
with an oxidant followed by a base to provide the compound of Formula IV.

8. The process of claim 7, further comprising preparing the compound of Formula V by a process comprising treating a compound of Formula VI 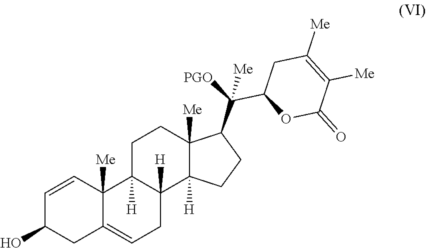
with 2-nitrophenyl selenocyanate in the presence of a phosphine to provide the compound of Formula V.

9. The process of claim 8, further comprising preparing the compound of Formula VI by a process comprising treating a compound of Formula VII 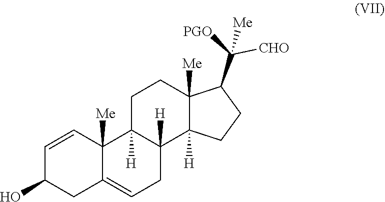
with a compound of Formula VIII 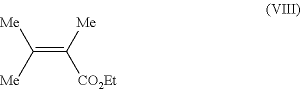
which has been previously treated with an amide base to provide the compound of Formula VI.

10. The process of claim 9, further comprising preparing the compound of Formula VII by a process comprising treating a compound of Formula IX 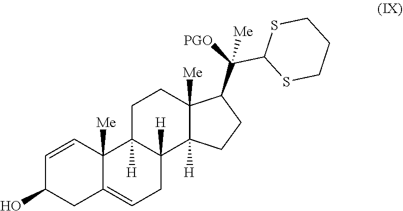
under reaction conditions suitable for conversion of the 1,3-dithiolane moiety in Formula IX into an aldehyde moiety to provide the compound of Formula VII.

11. The process of claim 10, further comprising preparing the compound of Formula IX by a process comprising treating a compound of Formula X 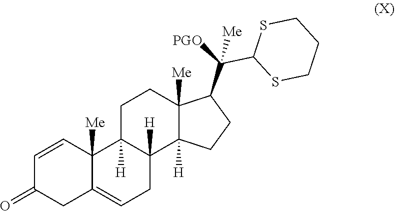
with sodium borohydride in the presence of cerium trichloride to provide the compound of Formula IX.

12. The process of claim 11, further comprising preparing the compound of Formula X by a process comprising treating a compound of Formula XI 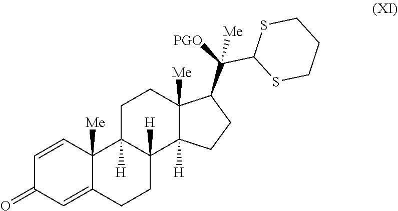
with an alkoxide base to provide the compound of Formula X.

13. The process of claim 12, further comprising preparing the compound of Formula XI by a process comprising treating a compound of Formula XII 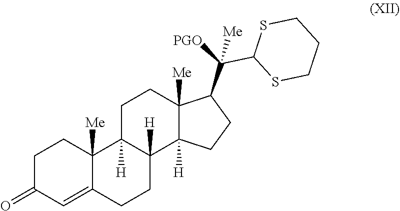
with an amide base followed by treating with N-tert-butyl phenylsulfinimidoyl chloride to provide the compound of Formula XI.

14. The process of claim 13, further comprising preparing the compound of Formula XII by a process comprising treating a compound of Formula XIII 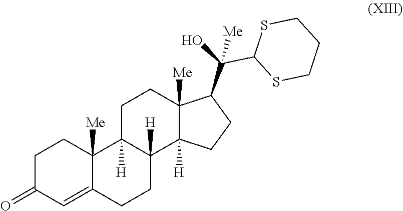
under suitable reaction conditions for introduction of a protection group PG to provide the compound of Formula XII.

15. The process of claim 14, further comprising preparing the compound of Formula XIII by a process comprising treating a compound of Formula XIV 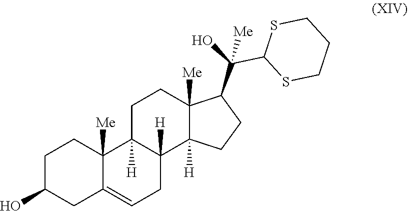
with aluminum isopropoxide to provide the compound of Formula XIII.

16. The process of claim 15, further comprising preparing the compound of Formula XIV by a process comprising treating Pregnenolone 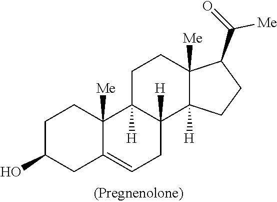
with 1,3-dithiane that has been previously treated with an organolithium base to provide the compound of Formula XIV.

17. Withanolide D prepared by a process of claim 1:

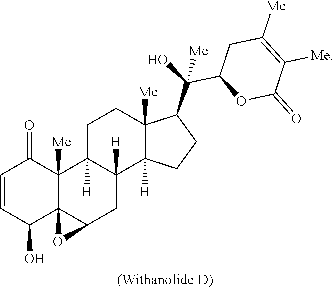

18. Withanolide A prepared by a process of claim 3:

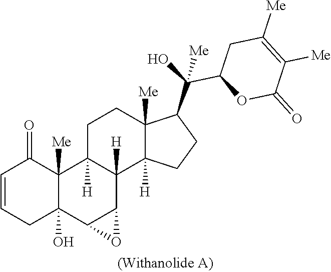

19. A process of preparing a compound of Formula A 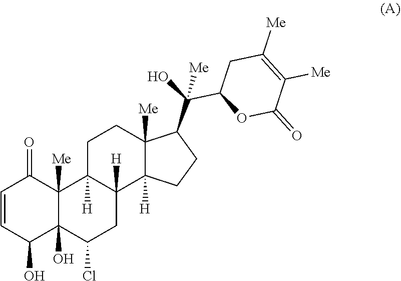
comprising treating Withanolide D 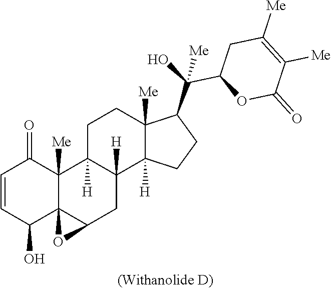
with cerium trichloride to provide the compound of Formula A.

20. A process of preparing a compound of Formula B 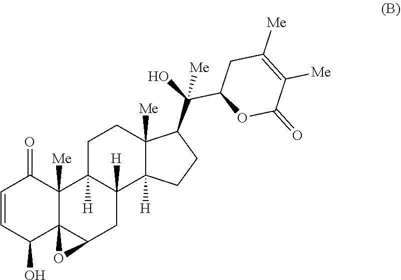
comprising treating Withanolide D 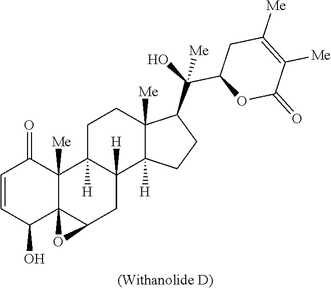
under hydrogenation conditions to provide the compound of Formula B.

21. A compound selected from: 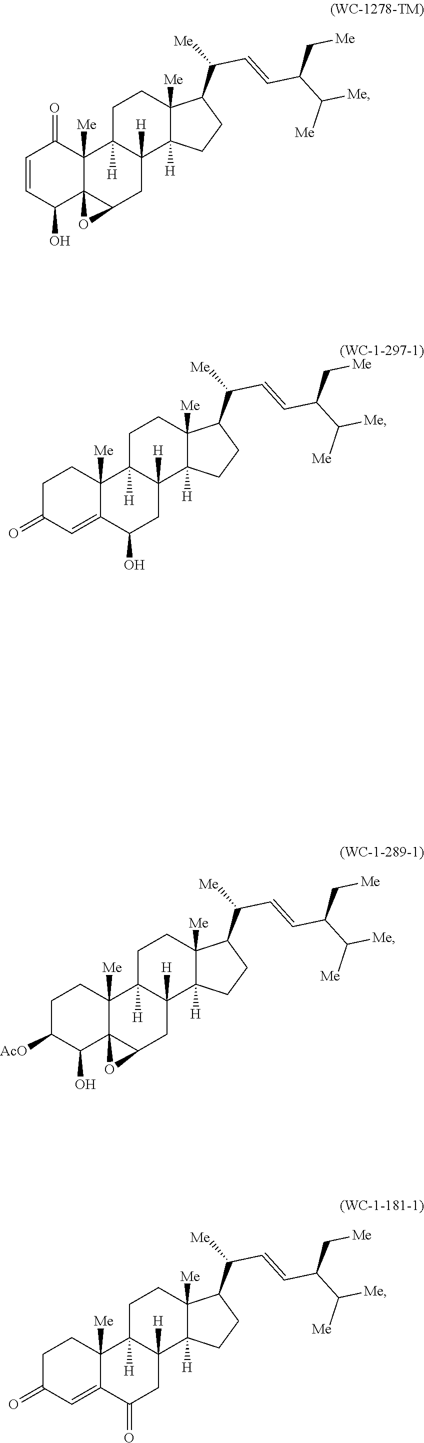 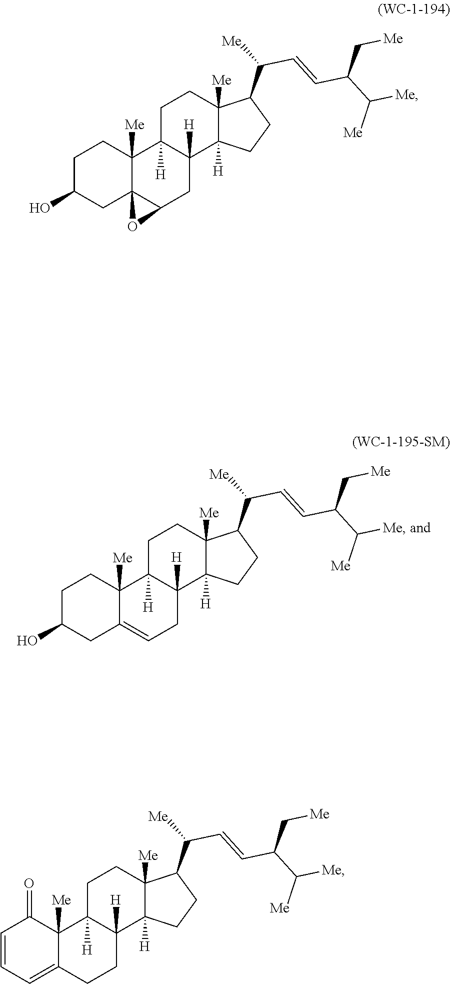
or a pharmaceutically acceptable salt or derivative thereof.
